(19) United States
(12) Patent Application Publication
(10) Pub. No.: US 2018/0057866 A1
(43) Pub. Date: Mar. 1, 2018

(54) GENETIC PROFILING METHODS FOR PREDICTION OF TASTE AND SCENT PREFERENCES AND GUSTATIVE AND OLFACTIVE PRODUCT SELECTION

(71) Applicants: Ron A. Andrews, Coto de Caza, CA (US); Shannon M. Kieran, Scottsdale, AZ (US); Sara Riordan, Roseville, CA (US); Elissa Levin, San Francisco, CA (US); Zack Miskel, Napa, CA (US); Josh Riggs, Trabuco Canyon, CA (US)

(72) Inventors: Ron A. Andrews, Coto de Caza, CA (US); Shannon M. Kieran, Scottsdale, AZ (US); Sara Riordan, Roseville, CA (US); Elissa Levin, San Francisco, CA (US); Zack Miskel, Napa, CA (US); Josh Riggs, Trabuco Canyon, CA (US)

(73) Assignee: Exploragen, Inc., Rancho Santa Margarita, CA (US)

(21) Appl. No.: 15/671,160

(22) Filed: Aug. 8, 2017

Related U.S. Application Data
(60) Provisional application No. 62/371,843, filed on Aug. 8, 2016.

(51) Int. Cl. C12Q 1/68

(52) U.S. Cl. CPC C12Q 1/6827; C12Q 2600/156; C12Q 1/6876

(57) ABSTRACT

The present invention relates to methods of gustative or olfactive product selection. In particular, the invention relates to the use of genetic profiling to predict individual taste and/or scent preferences for gustative or olfactive products and methods for selection of gustative or olfactive products for an individual based on such genetic profiling.

### RELATED APPLICATIONS

This application claims priority to U.S. Provisional Patent Application Ser. No. 62/371,843, filed on Aug. 8, 2016, which is hereby incorporated by reference in its entirety.

### FIELD OF THE INVENTION

The present invention relates to methods of gustative or olfactive product selection. In particular, the invention relates to the use of genetic profiling to predict individual taste and/or scent preferences for gustative or olfactive products and methods for selection of gustative or olfactive products for an individual based on such genetic profiling.

### BACKGROUND OF THE INVENTION

The large number of gustative (e.g., food and beverage) and olfactive (e.g., perfumes, colognes) products available and their great variation in taste or smell present a challenge and create uncertainty regarding which products will best suit the taste and scent preferences of an individual consumer. Moreover, the expansion of the food and beverage industry has been accompanied by increasing numbers of products, brands, and styles and is making ever more varieties of gustative and olfactive products available to the consumer.

Taste and smell are the most important factors for a consumer when purchasing a food, beverage, or fragrance product. However, consumers have different taste and scent preferences (e.g., sweet, sour, salty, bitter, savory, musk). For any individual consumer, certain tastes and scents may be preferred (e.g., sweet) while other tastes and scents will be rejected (e.g., sour).

Thus, there remains a need in the food, beverage, and fragrance industry for better methods of determining individual taste and/or scent preferences to enable selection of gustative and olfactive products that will satisfy the taste and/or scent preferences of an individual.

### SUMMARY OF THE INVENTION

The present invention relates to the use of genetic profiling in combination with questioning about personal characteristics and taste and/or scent preferences to predict individual taste and/or scent preferences for gustative and olfactive products. The methods of the invention can be used to provide gustative and olfactive products to consumers based on their predicted taste and/or scent preferences.

In some embodiments, the present invention provides a method for assaying a sample from a subject, the method comprising: a) obtaining a biological sample comprising nucleic acids from a subject; b) processing the sample to isolate or enrich the sample for the nucleic acids; and c) analyzing the genotype of the biological sample at a plurality of SNPs in a panel of SNPs identified as useful for identifying taste and/or scent preference. In other embodiments, the invention provides a method comprising: a) obtaining taste and/or scent preference information from a subject; b) obtaining a biological sample comprising nucleic acids from the subject; c) processing the sample to isolate or enrich the sample for the nucleic acids; and d) analyzing the genotype of the biological sample at a plurality of SNPs in a panel of SNPs identified as useful for identifying taste and/or scent preference. In some embodiments, the plurality of SNPs is selected from the group consisting of rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664. In some embodiments, the plurality of SNPs comprises rs3930459, rs42451, rs2708377, rs71443637, and rs12226920. In some embodiments, the plurality of SNPs comprises rs3930459 and rs42451. In some embodiments, the plurality of SNPs comprises rs3930459 and rs2708377. In some embodiments, the plurality of SNPs comprises rs3930459 and rs71443637. In some embodiments, the plurality of SNPs comprises rs3930459 and rs12226920. In some embodiments, the plurality of SNPs comprises rs42451 and rs2708377. In some embodiments, the plurality of SNPs comprises rs42451 and rs71443637. In some embodiments, the plurality of SNPs comprises rs42451 and rs12226920. In some embodiments, the plurality of SNPs comprises rs2708377 and rs71443637. In some embodiments, the plurality of SNPs comprises rs2708377 and rs12226920. In some embodiments, the plurality of SNPs comprises rs71443637 and rs12226920. In some embodiments, the plurality of SNPs comprises at least 5, 10, 15, 20, 25, 30, 35 or 41 SNPs selected from Table 1.

In one embodiment, the present invention provides a method comprising: a) obtaining a biological sample from a subject; and b) detecting the presence of a plurality of SNPs selected from the group consisting of rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664. In some embodiments, the plurality of SNPs comprises at least 2, 3, 4, 5, 10, 15, 20, 25, 30, 35 or 41 SNPs selected from Table 1.

In one embodiment, the present invention provides a method comprising: a) obtaining a biological sample from a subject; and b) detecting the presence of a plurality of targets selected from the group consisting of FGF21, FTO, OR10A6, OR10A2, OR10A4, OR10A3, OR6A2, CA6, TAS1R3, GNAT3, TAS2R19, OR2J3, OR2J3, OR5A1, CD36, TAS2R50, SCNN1D, ORD74, TRPA1, TAS2R60, TAS2R20, GNAT3, ORD74, TAS2R38, GHRL, TAS2R46, OR2J3, TAS1R3, TAS1R2, TRPV1, TAS2R43, HLA-DOA, TAS1R1, and TAS2R16. In some embodiments, the plurality of targets comprises OR10A6, OR10A2, OR10A4, OR10A3, OR6A2, GHRL, TAS2R46, TAS2R43, and TAS2R20. In some embodiments, the plurality of targets comprises OR10A6, GHRL, TAS2R46, TAS2R43, and TAS2R20. In some embodiments, the plurality of targets comprises OR10A2, GHRL, TAS2R46, TAS2R43, and TAS2R20. In some embodiments, the plurality of targets comprises OR10A4, GHRL, TAS2R46, TAS2R43, and TAS2R20. In some embodiments, the plurality of targets comprises OR10A3, GHRL, TAS2R46, TAS2R43, and TAS2R20. In some embodiments, the plurality of targets comprises OR6A2, GHRL, TAS2R46, TAS2R43, and TAS2R20. In some embodiments, the plurality of targets comprises GHRL and TAS2R46. In some embodiments, the plurality of targets comprises GHRL and TAS2R43. In some embodiments, the plurality of targets comprises GHRL and TAS2R20. In some embodiments, the plurality of targets comprises TAS2R46 and TAS2R43. In some embodiments, the plurality of targets comprises TAS2R46 and TAS2R20. In some embodiments, the plurality of targets comprises TAS2R43 and TAS2R20. In some embodiments, the plurality of targets comprises at least 2, 3, 4, 5, 10, 15, 20, 25, 30, 35 or 41 targets selected from Table 1.

In some embodiments, the present invention provides a method for predicting taste and/or scent preferences of a subject, the method comprising: a) providing a biological sample comprising DNA from the subject; b) genotyping a plurality of SNPs in the sample, wherein the plurality of SNPs comprises one or more SNPs identified as useful for identifying taste and/or scent preference; c) obtaining taste and/or scent preference information from the subject; and d) determining the subject's taste and/or scent preference scores for one or more food or beverage product, thereby predicting taste and/or scent preferences for the subject for a gustative or olfactive product. In some embodiments, the method further comprises: calculating gustative and/or olfactive product bin scores based on the genotyping from step (b), the information obtained about the subject from step (c), and the taste and/or scent preference scores for the one or more gustative or olfactive products from step (d). In some embodiments, the food or beverage product is selected from the group consisting of wine, liquor, beer, and coffee. In certain embodiments, the liquor is selected from the group consisting of gin, scotch whiskey, rum, and vodka.

In some aspects, the gustative or olfactive product bins comprise one or more gustative or olfactive product bins. In certain aspects, the gustative or olfactive bin scores are used to predict that the subject will prefer gustative or olfactive products from a gustative or olfactive product bin having a higher gustative or olfactive product bin score than a gustative or olfactive product bin having a lower gustative or olfactive product bin score. The method may be performed prior to introducing a gustative or olfactive product to the subject, prior to selling a gustative or olfactive product to the subject, or prior to recommending a gustative or olfactive product to the subject. In one embodiment, the method further comprises creating and storing a user taste and/or scent profile comprising information about the subject's gustative and olfactive product preferences based on the subject's gustative and/or olfactive product bin scores. In other embodiments, the method further comprises providing the subject with at least one sample of a gustative or olfactive product from the gustative or olfactive bin having the highest gustative or olfactive product bin score. Additionally the subject may be provided with one or more gustative and olfactive product samples from other gustative and/or olfactive product bins, preferably the top scoring gustative and/or olfactive product bins. For example, the subject may be provided with one or more gustative and/or olfactive samples from one or more of the gustative and/or olfactive product bins having the top two, three, or four gustative and/or olfactive product bin scores. In some embodiments, the food or beverage product is selected from the group consisting of wine, liquor, beer, and coffee. In certain embodiments, the liquor is selected from the group consisting of gin, scotch whiskey, rum, and vodka.

In other embodiments, the invention provides a method for providing a gustative and/or olfactive product to a subject, the method comprising the following steps: (a) providing a biological sample comprising DNA from the subject; (b) genotyping the sample to determine which alleles are present at a set of single nucleotide polymorphisms identified as useful for identifying taste and/or scent preference; (c) obtaining information about the subject; (d) determining the subject's taste and/or scent preference scores for one or more gustative and/or olfactive products; and (e) calculating gustative and/or olfactive product bin scores for one or more gustative and/or olfactive product bins based on the genotyping from step (b), the information obtained about the subject from step (c), and the taste and/or scent preference scores for one or more gustative and/or olfactive products from step (d); and (f) providing the subject with at least one gustative and/or olfactive product from the gustative and/or olfactive product bin that has the subject's highest gustative and/or olfactive product bin score. In some aspects, the information about the subject is selected from the group consisting of the subject's gender; the subject's age; and whether the subject prefers sweet, sour, salty, bitter, or savory foods. The method may be performed prior to serving a gustative and/or olfactive product to the subject, prior to selling a gustative and/or olfactive product to the subject, or prior to recommending a gustative and/or olfactive product to the subject. In one embodiment, the method further comprises creating and storing a user taste and/or scent profile comprising information about the subject's gustative and/or olfactive product preferences based on the subject's gustative and/or olfactive product bin scores. In other embodiments, the method further comprises providing the subject with at least one sample of gustative and/or olfactive product from the gustative and/or olfactive product bin having the highest gustative and/or olfactive product bin score. Additionally the subject may be provided with one or more gustative and/or olfactive product samples from other gustative and/or olfactive product bins, preferably the top scoring gustative and/or olfactive product bins. For example, the subject may be provided with one or more gustative and/or olfactive product samples from one or more of the gustative and/or olfactive product bins having the top two, three, or four gustative and/or olfactive product bin scores. In some embodiments, the food or beverage product is selected from the group consisting of wine, liquor, beer, and coffee. In certain embodiments, the liquor is selected from the group consisting of gin, scotch whiskey, rum, and vodka.

In some embodiments, the present invention provides a method of creating a taste and/or scent profile for a subject, the method comprising: a) obtaining a biological sample comprising nucleic acids from a subject; b) processing the sample to isolate or enrich the sample for the nucleic acids; and c) assaying a plurality of SNPs in the sample, wherein the plurality of SNPs comprises one or more SNPs identified as useful for identifying taste and/or scent preference, thereby creating a taste and/or scent profile for the subject.

In some embodiments, the present invention provides a method of creating a taste and/or scent profile for a subject, the method comprising: a) providing a biological sample comprising DNA from the subject; b) genotyping a plurality of SNPs in the sample, wherein the plurality of SNPs comprises one or more SNPs identified as useful for identifying taste and/or scent preference; c) obtaining taste and/or scent preference information from the subject; and d) determining the subject's taste and/or scent preference scores for one or more gustative and/or olfactive products, thereby predicting gustative and/or olfactive product preferences for the subject. In some embodiments, the method further comprises: calculating gustative and/or olfactive product bin scores based on the genotyping from step (b), the information obtained about the subject from step (c), and the taste and/or scent preference scores for the one or more gustative and/or olfactive products from step (d). In certain aspects, the gustative and/or olfactive product bin scores are used to predict that the subject will prefer gustative and/or olfactive product from a gustative and/or olfactive product bin having a higher gustative and/or olfactive product bin score than a gustative and/or olfactive product bin having a lower gustative and/or olfactive product bin score. In other embodiments, the method further comprises creating the taste and/or scent profile for the subject based on steps (a)-(d). In one embodiment, the method further comprises selecting a gustative and/or olfactive product for the subject based on the taste and/or scent profile. In another embodiment, the method further comprises storing the taste and/or scent profile. In some embodiments, the food or beverage product is selected from the group consisting of wine, liquor, beer, and coffee. In certain embodiments, the liquor is selected from the group consisting of gin, scotch whiskey, rum, and vodka.

In another aspect, the invention includes a method of creating a taste and/or scent profile for a subject, the method comprising: a) providing a biological sample comprising DNA from the subject; b) genotyping the sample to determine which alleles are present at one or more SNPs identified as useful for identifying taste and/or scent preference; c) obtaining information about the subject; d) determining the subject's taste and/or scent preference scores for one or more gustative and/or olfactive products; e) calculating gustative and/or olfactive product bin scores for one or more gustative and/or olfactive product bins based on the genotyping from step (b), the information obtained about the subject from step (c), and the taste and/or scent preference scores for the one or more gustative and/or olfactive products from step (d), wherein the gustative and/or olfactive product bin scores are used to predict that the subject will prefer gustative and/or olfactive products from a gustative and/or olfactive product bin having a higher gustative and/or olfactive product bin score than a gustative and/or olfactive product bin having a lower gustative and/or olfactive product bin score; and f) creating the taste and/or scent profile for the subject based on steps (a)-(e). In one embodiment, the method further comprises selecting a gustative and/or olfactive product for the subject based on the gustative and/or olfactive product profile. In another embodiment, the method further comprises storing the taste and/or scent profile. In some embodiments, the method further comprises selecting a gustative and/or olfactive product for the subject based on the user taste and/or scent profile. In other embodiments, the method further comprises storing the user taste and/or scent profile. In some embodiments, the food or beverage product is selected from the group consisting of wine, liquor, beer, and coffee. In certain embodiments, the liquor is selected from the group consisting of gin, scotch whiskey, rum, and vodka.

In another aspect the invention includes a method of providing a gustative and/or olfactive product to a subject, the method comprising the following steps: a) providing a biological sample comprising DNA from the subject; b) genotyping the sample to determine which alleles are present at single nucleotide polymorphisms identified as useful for identifying taste and/or scent preference; c) obtaining information about the subject; d) determining the subject's taste and/or scent preference scores for one or more gustative and/or olfactive products; and e) calculating gustative and/or olfactive product bin scores for one or more gustative and/or olfactive product bins based on the genotyping from step (b), the information obtained about the subject from step (c), and the taste and/or scent preference scores for one or more gustative and/or olfactive products from step (d); and f) providing the subject with at least one gustative and/or olfactive product from the gustative and/or olfactive product bin that has the subject's highest gustative and/or olfactive product bin score. In another embodiment, the method further comprises providing the subject with at least one gustative and/or olfactive product from a gustative and/or olfactive product bin that has a positive ranking. In certain embodiments, the method further comprises providing at least one gustative and/or olfactive product to the subject on a periodic basis. In another embodiment, the method further comprises providing the subject with a plurality of gustative and/or olfactive product samples from one or more of the gustative and/or olfactive product bins having positive rankings or preferably the top two, three, or four gustative and/or olfactive product bin scores. In some embodiments, the food or beverage product is selected from the group consisting of wine, liquor, beer, and coffee. In certain embodiments, the liquor is selected from the group consisting of gin, scotch whiskey, rum, and vodka.

Genotyping of the single nucleotide polymorphisms in the methods of the present invention can be performed using any method known in the art, including, but not limited to, dynamic allele-specific hybridization (DASH), microarray analysis, Tetra-primer ARMS-PCR, a TaqMan 5′-nuclease assay; an Invader assay with Flap endonuclease (FEN), a Serial Invasive Signal Amplification Reaction (SISAR), an oligonucleotide ligase assay, restriction fragment length polymorphism (RFLP), single-strand conformation polymorphism, temperature gradient gel electrophoresis (TGGE), denaturing high performance liquid chromatography (DHPLC), sequencing, and immunoassay.

In another aspect, the invention includes a computer implemented method for predicting taste and/or scent preferences of a subject, the computer performing steps comprising: a) receiving inputted data comprising: i) genotyping information for the subject regarding which alleles are present at one or more SNPs identified as useful for identifying taste and/or scent preference, ii) values for the subject's taste and/or scent preference scores for one or more gustative and/or olfactive products, and iii) information about the subject; b) calculating the subject's gustative and/or olfactive product bin scores for one or more gustative and/or olfactive product bins based on the subject's taste and/or scent preference scores for one or more gustative and/or olfactive products, wherein the gustative and/or olfactive product bin scores are used to predict that the subject will prefer gustative and/or olfactive products from a gustative and/or olfactive product bin having a higher gustative and/or olfactive product bin score than a gustative and/or olfactive product bin having a lower gustative and/or olfactive product bin score; and c) displaying information regarding predicted gustative and/or olfactive product preferences of the subject. In one embodiment, the computer implemented method further comprises storing a user profile for the subject comprising information regarding the subject's taste and/or scent preferences. In other embodiments, the method further comprises creating and storing a user taste and/or scent profile comprising information about the subject's taste and/or scent preferences based on the subject's gustative and/or olfactive product bin scores. In some embodiments, the food or beverage product is selected from the group consisting of wine, liquor, beer, and coffee. In certain embodiments, the liquor is selected from the group consisting of gin, scotch whiskey, rum, and vodka.

In certain embodiments, the computer implemented method further comprises inputting a list of gustative and/or olfactive products from a commercial establishment (e.g., restaurant, winery, bar, or store), and displaying a listing of gustative and/or olfactive products available at the commercial establishment that belong to the gustative and/or olfactive product bin having the highest gustative and/or olfactive product bin score. The list of gustative and/or olfactive products from a commercial establishment may be obtained, for example, by providing an image of a menu from the commercial establishment, and processing the image to obtain the list of gustative and/or olfactive products. Alternatively, the list of gustative and/or olfactive products may be obtained by identifying the commercial establishment, and retrieving an electronic representation of the list of gustative and/or olfactive products from a gustative and/or olfactive product list database. In certain embodiments, the computer implemented method further comprises displaying a listing of gustative and/or olfactive products available at a commercial establishment that belong to the gustative and/or olfactive product bins having the top two, three, or four gustative and/or olfactive product bin scores. In another embodiment, the computer implemented method further comprises displaying a listing of gustative and/or olfactive products available at a commercial establishment that belong to the gustative and/or olfactive product bins having positive rankings. In some embodiments, the food or beverage product is selected from the group consisting of wine, liquor, beer, and coffee. In certain embodiments, the liquor is selected from the group consisting of gin, scotch whiskey, rum, and vodka.

In another aspect, the invention includes a system for performing the computer implemented method for predicting taste and/or scent preferences of a subject, as described herein. The system comprises: a) a storage component for storing data, wherein the storage component has instructions for determining the taste and/or scent preferences of the subject stored therein; b) a computer processor for processing data, wherein the computer processor is coupled to the storage component and configured to execute the instructions stored in the storage component in order to receive data regarding the subject and analyze data according to one or more algorithms; and c) a display component for displaying information regarding the taste and/or scent preferences of the subject.

In another aspect, the invention includes a kit for creating a user taste and/or scent profile, the kit comprising at least one agent for determining which alleles are present at one or more SNPs identified as useful for identifying taste and/or scent preference. In certain embodiments, the kit comprises a set of allele-specific probes that hybridize to nucleic acids comprising one or more SNPs identified as useful for identifying taste and/or scent preference. In another embodiment, the kit comprises a SNP array comprising a set of allele-specific probes that hybridize to nucleic acids comprising one or more SNPs identified as useful for identifying taste and/or scent preference. In a further embodiment, the kit comprises a set of allele-specific primers for determining which alleles are present at one or more SNPs identified as useful for identifying taste and/or scent preference. Additionally, the kit may further comprise reagents for performing dynamic allele-specific hybridization (DASH), Tetra-primer ARMS-PCR, a TaqMan 5′-nuclease assay; an Invader assay with Flap endonuclease (FEN), a Serial Invasive Signal Amplification Reaction (SISAR), an oligonucleotide ligase assay, restriction fragment length polymorphism (RFLP), single-strand conformation polymorphism, temperature gradient gel electrophoresis (TGGE), denaturing high performance liquid chromatography (DHPLC), sequencing, or an immunoassay. The kit may further comprise samples of gustative and/or olfactive products. Additionally, the kit may further comprise a questionnaire comprising questions or a survey. The kit may further comprise information, in electronic or paper form, comprising instructions on how to determine taste and/or scent preferences of a subject. In some embodiments, the food or beverage product is selected from the group consisting of wine, liquor, beer, and coffee. In certain embodiments, the liquor is selected from the group consisting of gin, scotch whiskey, rum, and vodka.

In another aspect, the invention includes a set of allele-specific probes and/or primers for detecting which alleles are present at one or more SNPs identified as useful for identifying taste and/or scent preference. In another embodiment, the allele-specific probes are provided by a SNP array.

The methods of the invention make possible targeted marketing and distribution of gustative and/or olfactive products likely to please consumers. In some embodiments, the gustative and/or olfactive product is selected from the group consisting of wine, liquor, beer, and coffee. In certain embodiments, the liquor is selected from the group consisting of gin, scotch whiskey, rum, and vodka.

These and other embodiments of the subject invention will readily occur to those of skill in the art in view of the disclosure herein.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIG. 1 shows hierarchical clustering of wine preferences.

FIG. 2 shows relationships between wine preferences, also gauged using consensus clustering, which allowed the determination of a reduced number of dimensions that could be used to describe wine preference.

FIG. 3A shows hierarchical clustering of taste perception within twelve wines. FIG. 3B shows clustering of wine preference and genetic variants.

FIG. 4 shows the relationship between the model score and the actual wine preference.

FIG. 5 shows a schematic of the method for assigning wines to a subject.

FIG. 6 shows distribution of model scores for preference of gin.

FIG. 7 shows distribution of model scores for preference for cocktails.

FIG. 8 shows distribution of model scores for preference for scotch whiskey.

FIG. 9 shows distribution of model scores for preference dark beer.

FIG. 10 shows distribution of model scores for preference for IPA beer.

FIG. 11 shows distribution of model scores for preference for rum.

FIG. 12 shows distribution of model scores for preference for vodka.

FIG. 13 shows distribution of model scores for preference for black coffee.

### DETAILED DESCRIPTION OF THE INVENTION

The practice of the present invention will employ, unless otherwise indicated, conventional methods of genetics, biochemistry, molecular biology and recombinant DNA techniques, within the skill of the art. Such techniques are explained fully in the literature. See, e.g., Single Nucleotide Polymorphisms: Methods and Protocols (Methods in Molecular Biology, A. A. Komar ed., Humana Press; 2^{nd} edition, 2009); Genetic Variation: Methods and Protocols (Methods in Molecular Biology, M. R. Barnes and G. Breen eds., Humana Press, 2010); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

### I. Definitions

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a nucleic acid” includes a mixture of two or more such nucleic acids, and the like.

The terms “polymorphism,” “polymorphic nucleotide,” “polymorphic site” or “polymorphic nucleotide position” refer to a position in a nucleic acid that possesses the quality or character of occurring in several different forms. A nucleic acid polymorphism is characterized by two or more “alleles,” or versions of the nucleic acid sequence. Typically, an allele of a polymorphism that is identical to a reference sequence is referred to as a “reference allele” and an allele of a polymorphism that is different from a reference sequence is referred to as an “alternate allele,” or sometimes a “variant allele.” As used herein, the term “major allele” refers to the more frequently occurring allele at a given polymorphic site, and “minor allele” refers to the less frequently occurring allele, as present in the general or study population.

The term “single nucleotide polymorphism” or “SNP” refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. Single nucleotide polymorphisms can also arise from a deletion of a nucleotide or an insertion of a nucleotide relative to a reference allele.

SNPs generally are described as having a minor allele frequency, which can vary between populations, but generally refers to the sequence variation (A, T, G, or C) that is less common than the major allele. The frequency can be obtained from dbSNP or other sources, or may be determined for a certain population using Hardy-Weinberg equilibrium (See for details see Eberle M A, Rieder M J, Kruglyak L, Nickerson D A (2006) Allele Frequency Matching Between SNPs Reveals an Excess of Linkage Disequilibrium in Genic Regions of the Human Genome. PLoS Genet 2(9): e142. doi:10.1371/journal.pgen.0020142; herein incorporated by reference).

Further details of the SNPs described here can be found in the National Center for Biotechnology Information (NCBI) SNP database, available online at ncbi.nlm.nih.gov/projects/SNP, from which the following brief descriptions are taken. Population data on these SNPs may be found at this site. The SNP representations are according to the NCBI SNP database conventions, and show the two alleles between brackets.

As used herein, the term “probe” refers to a polynucleotide that contains a nucleic acid sequence complementary to a nucleic acid sequence present in the target nucleic acid analyte (e.g., at SNP location). The polynucleotide regions of probes may be composed of DNA, and/or RNA, and/or synthetic nucleotide analogs. Probes may be labeled in order to detect the target sequence. Such a label may be present at the 5′ end, at the 3′ end, at both the 5′ and 3′ ends, and/or internally.

An “allele-specific probe” hybridizes to only one of the possible alleles of a SNP under suitably stringent hybridization conditions.

The term “primer” as used herein, refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is preferably single-stranded for maximum efficiency in amplification, but may alternatively be double-stranded. If double-stranded, the primer can first be treated to separate its strands before being used to prepare extension products. This denaturation step is typically affected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis. Typically, nucleic acids are amplified using at least one set of oligonucleotide primers comprising at least one forward primer and at least one reverse primer capable of hybridizing to regions of a nucleic acid flanking the portion of the nucleic acid to be amplified.

An “allele-specific primer” matches the sequence exactly of only one of the possible alleles of a SNP, hybridizes at the SNP location, and amplifies only one specific allele if it is present in a nucleic acid amplification reaction.

As used herein, the term “user” or “consumer” may refer to any individual person, group of people, or association that purchases, uses, or consumes gustative and/or olfactive products. A user taste and/or scent profile may include a ranking of gustative and/or olfactive products based on gustative and/or olfactive product bin scores derived from SNP genotyping and querying subjects on personal characteristics and taste and/or scent preferences, as described herein.

An “odor” is any scent or smell, whether pleasant or offensive. An odor is consciously perceived by an individual when odorant molecules bind to the olfactory epithelium of the nasal passage. An “odorant” is an odorous substance. Perfumery materials, whether natural or synthetic, are described as odorants. A “perfumery odorant” is an odorant used for the principal purpose of providing an odor. A “scent” is the odor left behind by an animal or individual. People use perfumes to augment their natural scent.

A “perfume” or a “fragrance composition” is a specific pleasantly odorous cosmetic composition for topical application to an individual. Technically, perfumes are mixtures of a variety of substances, and may include natural materials of vegetable or animal origin, wholly or partly artificial compounds, or mixtures thereof. Dissolved in alcohol, these mixtures of various volatile fragrant substances release their scents into the air at normal temperatures. To a perfumer, only the extrait—the mixture which contains the highest proportion of fragrance concentrate and the least possible alcohol—is called perfume. Mixtures of lower concentration include eau de parfum, after shave, eau de toilette, eau de sport, splash cologne, eau de cologne, cologne, eau fraiche, and the like. In addition to the fragrance solutions which are diluted with alcohol, there are also those which are diluted with oil. Furthermore, compact and cream perfumes are produced by mixing up to 25% fragrance oil with solids such as paraffin or other waxes.

A “pheromone” is a biochemical produced by an animal or individual which elicits a specific physiological or behavioral response in another member of the same species. In addition to physiological responses, pheromones can be identified by their species specific binding to receptors in the vomeronasal organ (VNO). The binding of pheromones is generally sexually dimorphic. Naturally occurring human pheromones induce sexually dimorphic changes in receptor binding potential in vivo in the human VNO.

“Sexually dimorphic” refers to a difference in the effect of, or response to, a compound or composition between males and females of the same species.

The “vomeronasal organ” is a cul-de-sac which opens to the nasal passage and contains specialized receptor cells for pheromones.

### II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery of SNPs that can be used to determine gustative and/or olfactive products taste and/or scent preferences of individuals. Genetic profiling of an individual to identify the alleles present at these SNPs is used in combination with specific questioning about personal characteristics and taste and/or scent preferences in creating a taste and/or scent profile for an individual. The methods of the invention can be used for selecting and providing gustative and/or olfactive products to a consumer that have a high probability of meeting the individual's taste and/or scent preferences.

Detecting and Genotyping Gene Polymorphisms Associated with Taste and/or Scent Preferences

The SNPs of the present invention can be used as genetic markers for determining taste and/or scent preferences of an individual. These genetic markers can be used in combination with gustative and/or olfactive product sampling of a set of typifying gustative and/or olfactive products (i.e. representing different types, varieties, or styles of gustative and/or olfactive products) and querying the individual regarding certain personal characteristics and taste and/or scent preferences to create a user profile for an individual comprising information regarding likely taste and/or scent preferences for different gustative and/or olfactive products. SNPs that may be used in the methods and panels of the present invention include rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664. Generally, the methods of the present invention comprise: a) obtaining a biological sample comprising nucleic acids from a subject; b) processing the sample to isolate or enrich the sample for the nucleic acids; and c) analyzing the genotype of the biological sample at a plurality of SNPs in a panel of SNPs identified as useful for identifying taste and/or scent preference. In some instances the plurality of SNPs comprises one or more SNPs selected from Table 1. In some instances, the plurality of targets comprises at least about 2, at least about 3, at least about 4, at least about 5, at least about 6, at least about 7, at least about 8, at least about 9, at least about 10, at least about 15, at least about 20, at least about 30, at least about 40, or at least about 41 SNPs from Table 1.

For genetic testing, a biological sample containing nucleic acids is collected from an individual. The biological sample is typically saliva or cells from buccal swabbing, but can be any sample from bodily fluids, tissue or cells that contains genomic DNA or RNA of the individual. In certain embodiments, nucleic acids from the biological sample are isolated, purified, and/or amplified prior to analysis using methods well-known in the art. See, e.g., Green and Sambrook Molecular Cloning: A Laboratory Manual (Cold Spring Harbor Laboratory Press; 4^{th} edition, 2012); and Current Protocols in Molecular Biology (Ausubel ed., John Wiley & Sons, 1995); herein incorporated by reference in their entireties.

SNPs can be detected in a sample by any suitable method known in the art. Detection of a SNP can be direct or indirect. For example, the SNP itself can be detected directly. Alternatively, the SNP can be detected indirectly from cDNAs, amplified RNAs or DNAs, or proteins expressed by the SNP allele. Any method that detects a single base change in a nucleic acid sample can be used. For example, allele-specific probes that specifically hybridize to a nucleic acid containing the polymorphic sequence can be used to detect SNPs. A variety of nucleic acid hybridization formats are known to those skilled in the art. For example, common formats include sandwich assays and competition or displacement assays. Hybridization techniques are generally described in Hames, and Higgins “Nucleic Acid Hybridization, A Practical Approach,” IRL Press (1985); Gall and Pardue, Proc. Natl. Acad. Sci. U.S.A., 63:378-383 (1969); and John et al Nature, 223:582-587 (1969).

Sandwich assays are commercially useful hybridization assays for detecting or isolating nucleic acids. Such assays utilize a “capture” nucleic acid covalently immobilized to a solid support and a labeled “signal” nucleic acid in solution. The clinical sample will provide the target nucleic acid. The “capture” nucleic acid and “signal” nucleic acid probe hybridize with the target nucleic acid to form a “sandwich” hybridization complex.

In one embodiment, the allele-specific probe is a molecular beacon. Molecular beacons are hairpin shaped oligonucleotides with an internally quenched fluorophore. Molecular beacons typically comprise four parts: a loop of about 18-30 nucleotides, which is complementary to the target nucleic acid sequence; a stem formed by two oligonucleotide regions that are complementary to each other, each about 5 to 7 nucleotide residues in length, on either side of the loop; a fluorophore covalently attached to the 5′ end of the molecular beacon, and a quencher covalently attached to the 3′ end of the molecular beacon. When the beacon is in its closed hairpin conformation, the quencher resides in proximity to the fluorophore, which results in quenching of the fluorescent emission from the fluorophore. In the presence of a target nucleic acid having a region that is complementary to the strand in the molecular beacon loop, hybridization occurs resulting in the formation of a duplex between the target nucleic acid and the molecular beacon. Hybridization disrupts intramolecular interactions in the stem of the molecular beacon and causes the fluorophore and the quencher of the molecular beacon to separate resulting in a fluorescent signal from the fluorophore that indicates the presence of the target nucleic acid sequence.

For SNP detection, the molecular beacon is designed to only emit fluorescence when bound to a specific allele of a SNP. When the molecular beacon probe encounters a target sequence with as little as one non-complementary nucleotide, the molecular beacon preferentially stay in its natural hairpin state and no fluorescence is observed because the fluorophore remains quenched. See, e.g., Nguyen et al. (2011) Chemistry 17(46):13052-13058; Sato et al. (2011) Chemistry 17(41):11650-11656; Li et al. (2011) Biosens Bioelectron. 26(5):2317-2322; Guo et al. (2012) Anal. Bioanal. Chem. 402(10):3115-3125; Wang et al. (2009) Angew. Chem. Int. Ed. Engl. 48(5):856-870; and Li et al. (2008) Biochem. Biophys. Res. Commun. 373(4):457-461; herein incorporated by reference in their entireties.

In another embodiment, detection of the SNP sequence is performed using allele-specific amplification. In the case of PCR, amplification primers can be designed to bind to a portion of one of the disclosed genes, and the terminal base at the 3′ end is used to discriminate between the major and minor alleles or mutant and wild-type forms of the genes. If the terminal base matches the major or minor allele, polymerase-dependent three prime extension can proceed. Amplification products can be detected with specific probes. This method for detecting point mutations or polymorphisms is described in detail by Sommer et al. in Mayo Clin. Proc. 64:1361-1372 (1989).

Tetra-primer ARMS-PCR uses two pairs of primers that can amplify two alleles of a SNP in one PCR reaction. Allele-specific primers are used that hybridize at the SNP location, but each matches perfectly to only one of the possible alleles. If a given allele is present in the PCR reaction, the primer pair specific to that allele will amplify that allele, but not the other allele of the SNP. The two primer pairs for the different alleles may be designed such that their PCR products are of significantly different length, which allows them to be distinguished readily by gel electrophoresis. See, e.g., Mufioz et al. (2009) J. Microbiol. Methods. 78(2):245-246 and Chiapparino et al. (2004) Genome. 47(2):414-420; herein incorporated by reference.

SNPs may also be detected by ligase chain reaction (LCR) or ligase detection reaction (LDR). The specificity of the ligation reaction is used to discriminate between the major and minor alleles of the SNP. Two probes are hybridized at the SNP polymorphic site of a nucleic acid of interest, whereby ligation can only occur if the probes are identical to the target sequence. See e.g., Psifidi et al. (2011) PLoS One 6(1):e14560; Asari et al. (2010) Mol. Cell. Probes. 24(6):381-386; Lowe et al. (2010) Anal Chem. 82(13):5810-5814; herein incorporated by reference.

SNPs can also be detected in a biological sample by sequencing and SNP typing. In the former method, one simply carries out whole genome sequencing of a DNA sample, and uses the results to detect the present sequences. Whole genome analysis is used in the field of “personal genomics,” and genetic testing services exist, which provide full genome sequencing using massively parallel sequencing. Massively parallel sequencing is described e.g. in U.S. Pat. No. 5,695,934, entitled “Massively parallel sequencing of sorted polynucleotides,” and US 2010/0113283 A1, entitled “Massively multiplexed sequencing.” Massively parallel sequencing typically involves obtaining DNA representing an entire genome, fragmenting it, and obtaining millions of random short sequences, which are assembled by mapping them to a reference genome sequence.

Commercial services are also available that are capable of genotyping approximately 1 million SNPs for a fixed fee. SNP analysis can be carried out with a variety of methods that do not involve massively parallel random sequencing. For example, a commercially available MassARRAY system can be used. This system uses matrix-assisted laser desorption ionization time-of-flight mass spectrometry (MALDI-TOF MS) coupled with single-base extension PCR for high-throughput multiplex SNP detection. Another commercial SNP system, the Illumina Golden Gate assay, generates SNP specific PCR products that are subsequently hybridized to beads either on a solid matrix or in solution. Three oligonucleotides are synthesized for each SNP: two allele specific oligonucleotides (ASOs) that distinguish the SNP, and a locus specific sequence (LSO) just downstream of the SNP. The ASO and LSO sequences also contain target sequences for a set of universal primers (P1 through P3 in the adjacent figure), while each LSO also contains a particular address sequences (the “illumicode”) complementary to sequences attached to beads.

As another example, a SNP array comprising probes for detecting SNPs can be used. For example, SNP arrays are commercially available from Affymetrix and Illumina, which use multiple sets of short oligonucleotide probes for detecting known SNPs. The design of SNP arrays, such as manufactured by Affymetrix or Illumina, is described further in LaFamboise, “Single nucleotide polymorphism arrays: a decade of biological, computational and technological advances,” Nuc. Acids Res. 37(13):4181-4193 (2009).

Another method useful in SNP analysis is PCR-dynamic allele specific hybridization (DASH), which involves dynamic heating and coincident monitoring of DNA denaturation, as disclosed by Howell et al. (Nat. Biotech. 17:87-88, 1999). A target sequence is amplified (e.g., by PCR) using one biotinylated primer. The biotinylated product strand is bound to a streptavidin-coated microtiter plate well (or other suitable surface), and the non-biotinylated strand is rinsed away with alkali wash solution. An oligonucleotide probe, specific for one allele (e.g., the wild-type allele), is hybridized to the target at low temperature. This probe forms a duplex DNA region that interacts with a double strand-specific intercalating dye. When subsequently excited, the dye emits fluorescence proportional to the amount of double-stranded DNA (probe-target duplex) present. The sample is then steadily heated while fluorescence is continually monitored. A rapid fall in fluorescence indicates the denaturing temperature of the probe-target duplex. Using this technique, a single-base mismatch between the probe and target results in a significant lowering of melting temperature (Tm) that can be readily detected.

A variety of other techniques can be used to detect polymorphisms, including but not limited to, the Invader assay with Flap endonuclease (FEN), the Serial Invasive Signal Amplification Reaction (SISAR), the oligonucleotide ligase assay, restriction fragment length polymorphism (RFLP), single-strand conformation polymorphism, temperature gradient gel electrophoresis (TGGE), and denaturing high performance liquid chromatography (DHPLC). See, for example Molecular Analysis and Genome Discovery (R. Rapley and S. Harbron eds., Wiley 1^{st} edition, 2004); Jones et al. (2009) New Phytol. 183(4):935-966; Kwok et al. (2003) Curr Issues Mol. Biol. 5(2):43-60; Mufioz et al. (2009) J. Microbiol. Methods. 78(2):245-246; Chiapparino et al. (2004) Genome. 47(2):414-420; Olivier (2005) Mutat Res. 573(1-2):103-110; Hsu et al. (2001) Clin. Chem. 47(8):1373-1377; Hall et al. (2000) Proc. Natl. Acad. Sci. U.S.A. 97(15):8272-8277; Li et al. (2011) J. Nanosci. Nanotechnol. 11(2):994-1003; Tang et al. (2009) Hum. Mutat. 30(10):1460-1468; Chuang et al. (2008) Anticancer Res. 28(4A):2001-2007; Chang et al. (2006) BMC Genomics 7:30; Galeano et al. (2009) BMC Genomics 10:629; Larsen et al. (2001) Pharmacogenomics 2(4):387-399; Yu et al. (2006) Curr. Protoc. Hum. Genet. Chapter 7: Unit 7.10; Lilleberg (2003) Curr. Opin. Drug Discov. Devel. 6(2):237-252; and U.S. Pat. Nos. 4,666,828; 4,801,531; 5,110,920; 5,268,267; 5,387,506; 5,691,153; 5,698,339; 5,736,330; 5,834,200; 5,922,542; and 5,998,137 for a description of such methods; herein incorporated by reference in their entireties.

If the polymorphism is located in the coding region of a gene of interest, the SNP can be identified indirectly by detection of the variant protein produced by the SNP. Variant proteins (i.e., containing an amino acid substitution encoded by the SNP) can be detected using antibodies specific for the variant protein. For example, immunoassays that can be used to detect variant proteins produced by SNPs include, but are not limited to, immunohistochemistry (IHC), western blotting, enzyme-linked immunosorbent assay (ELISA), radioimmunoassays (RIA), “sandwich” immunoassays, fluorescent immunoassays, and immunoprecipitation assays, the procedures of which are well known in the art (see, e.g., Schwarz et al. (2010) Clin. Chem. Lab. Med. 48(12):1745-1749; The Immunoassay Handbook (D. G. Wild ed., Elsevier Science; 3^{rd} edition, 2005); Ausubel et al, eds, 1994, Current Protocols in Molecular Biology, Vol. 1 (John Wiley & Sons, Inc., New York); Coligan Current Protocols in Immunology (1991); Harlow & Lane, Antibodies: A Laboratory Manual (1988); Handbook of Experimental Immunology, Vols. I-IV (D. M. Weir and C. C. Blackwell eds., Blackwell Scientific Publications); herein incorporated by reference herein in their entireties).

### Sets of Allele-Specific Probes and Primers

The present invention also provides a probe set for predicting taste and/or scent preferences of a subject comprising a plurality of allele-specific probes for detecting which alleles are present at one or more SNPs identified as useful for identifying taste and/or scent preference.

The probe set may comprise one or more allele-specific polynucleotide probes. An allele-specific probe hybridizes to only one of the possible alleles of a SNP under suitably stringent hybridization conditions. Individual polynucleotide probes comprise a nucleotide sequence derived from the nucleotide sequence of the target SNP sequences or complementary sequences thereof. The nucleotide sequence of the polynucleotide probe is designed such that it corresponds to, or is complementary to the target SNP sequences. The allele-specific polynucleotide probe can specifically hybridize under either stringent or lowered stringency hybridization conditions to a region of the target sequences, to the complement thereof, or to a nucleic acid sequence (such as a cDNA) derived therefrom.

The selection of the allele-specific polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In one embodiment of the invention, the allele-specific polynucleotide probe is complementary to the polymorphic region of a single SNP allele target DNA or mRNA sequence. Computer programs can also be employed to select allele-specific probe sequences that may not cross hybridize or may not hybridize non-specifically.

The allele-specific polynucleotide probes of the present invention may range in length from about 15 nucleotides to the full length of the coding target or non-coding target. In one embodiment of the invention, the polynucleotide probes are at least about 15 nucleotides in length. In another embodiment, the polynucleotide probes are at least about 20 nucleotides in length. In a further embodiment, the polynucleotide probes are at least about 25 nucleotides in length. In another embodiment, the polynucleotide probes are between about 15 nucleotides and about 500 nucleotides in length. In other embodiments, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the probes are at least 15 nucleotides in length. In some embodiments, the probes are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The allele-specific polynucleotide probes of a probe set can comprise RNA, DNA, RNA or DNA mimetics, or combinations thereof, and can be single-stranded or double-stranded. Thus the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability. The probe set may comprise a coding target and/or a non-coding target. Preferably, the probe set comprises a combination of a coding target and non-coding target.

In another embodiment, the invention provides a set of allele-specific primers for detecting which alleles are present at one or more SNPs identified as useful for identifying taste and/or scent preference. An allele-specific primer matches the sequence exactly of only one of the possible alleles of a SNP, hybridizes at the SNP location, and amplifies only one specific allele if it is present in a nucleic acid amplification reaction. For use in amplification reactions such as PCR, a pair of primers can be used for detection of a SNP sequence. Each primer is designed to hybridize selectively to a single allele at the site of the SNP under stringent conditions, particularly under conditions of high stringency, as known in the art. The pairs of allele-specific primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays for SNP genotyping of subjects or biological samples from said subjects. Alternatively, these primers may be used in combination with probes, such as molecular beacons in amplifications using real-time PCR.

A label can optionally be attached to or incorporated into an allele-specific probe or primer polynucleotide to allow detection and/or quantitation of a target SNP polynucleotide. The target SNP polynucleotide may be from genomic DNA, expressed RNA, a cDNA copy thereof, or an amplification product derived therefrom, and may be the positive or negative strand, so long as it can be specifically detected in the assay being used. Similarly, an antibody may be labeled that detects a polypeptide expression product of the SNP allele.

In certain multiplex formats, labels used for detecting different SNPs may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the invention described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

The polynucleotides may be provided in a variety of formats, including as solids, in solution, or in an array. The polynucleotides may optionally comprise one or more labels, which may be chemically and/or enzymatically incorporated into the polynucleotide.

In some embodiments, one or more polynucleotides provided herein can be provided on a substrate. The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof. Conducting polymers and photoconductive materials can be used.

The substrate can take the form of an array, a photodiode, an optoelectronic sensor such as an optoelectronic semiconductor chip or optoelectronic thin-film semiconductor, or a biochip. The location(s) of probe(s) on the substrate can be addressable; this can be done in highly dense formats, and the location(s) can be microaddressable or nanoaddressable.

### SNP Arrays

The present invention contemplates that a set of allele-specific probes may be provided in an array format. In the context of the present invention, an “array” is a spatially or logically organized collection of polynucleotide probes. An array comprising probes specific for a coding target, non-coding target, or a combination thereof may be used. In some embodiments, an array is used which comprises a wide range of allele-specific probes for detecting one or more SNPs identified as useful for identifying taste and/or scent preference.

Typically the allele-specific polynucleotide probes are attached to a solid substrate and are ordered so that the location (on the substrate) and the identity of each are known. The allele-specific polynucleotide probes can be attached to one of a variety of solid substrates capable of withstanding the reagents and conditions necessary for use of the array. Examples include, but are not limited to, polymers, such as (poly)tetrafluoroethylene, (poly)vinylidenedifluoride, polystyrene, polycarbonate, polypropylene and polystyrene; ceramic; silicon; silicon dioxide; modified silicon; (fused) silica, quartz or glass; functionalized glass; paper, such as filter paper; diazotized cellulose; nitrocellulose filter; nylon membrane; and polyacrylamide gel pad. Substrates that are transparent to light are useful for arrays that may be used in an assay that involves optical detection.

Examples of array formats include membrane or filter arrays (for example, nitrocellulose, nylon arrays), plate arrays (for example, multiwell, such as a 24-, 96-, 256-, 384-, 864- or 1536-well, microtitre plate arrays), pin arrays, and bead arrays (for example, in a liquid “slurry”). Arrays on substrates such as glass or ceramic slides are often referred to as chip arrays or “chips.” Such arrays are well known in the art.

### Querying Subjects

Genotyping can be combined with personal information obtained by querying an individual regarding certain personal characteristics and taste and/or scent preferences to create a user profile comprising information regarding likely taste and/or scent preferences for different gustative and/or olfactive products.

Subjects are provided with a survey (i.e., questionnaire) comprising questions regarding the subject's characteristics and taste and/or scent preferences.

The survey questions may be provided in various formats, for example, orally (e.g., interviewing the subject), in writing (e.g., paper or electronic form), or through a website with interactive tools that guide the subject through the screening process.

Gustative and/or Olfactive Product Sampling

In addition, subjects whose gustative and/or olfactive product preferences are to be determined may be offered samples of typifying gustative and/or olfactive products representing different types, varieties, or styles of gustative and/or olfactive products and questioned regarding their preferences among these gustative and/or olfactive products.

In some embodiments, the gustative and/or olfactive products used in the methods of the present invention include natural and synthetic gustative and/or olfactive products. In some embodiments, the gustative and/or olfactive products used in the methods of the present invention include, but are not limited to, foods (e.g., chocolates), beverages (e.g., flavored drinks, alcoholic beverages, cocktails, teas, coffee), perfumes, natural odorants, synthetic odorants, pheromones, and sauces.

In some embodiments, the food or beverage product is wine, liquor, beer, or coffee. In certain embodiments, the liquor is selected from the group consisting of gin, scotch whiskey, rum, and vodka.

In some embodiments, the subject is offered one or more gustative and/or olfactive products for each gustative and/or olfactive product category to sample and questioned regarding taste and/or scent preferences. For each of the typifying gustative and/or olfactive products, taste and/or scent preferences are recorded, encoded as −1, representing “dislike”, 0, representing “no preference”, and +1, representing “like” the gustative and/or olfactive product.

Characterizing Gustative and/or Olfactive Products

In some embodiments, the gustative and olfactive products are characterized, analyzed, and cataloged for use in the methods of the present invention. Gas chromatography mass spectrometry (GC-MS) may be used to separate, identify, and quantify complex mixtures of chemicals in the gustative and olfactive products. The resulting GC-MS data may be used to catalog the products used in the methods of the present invention and link taste and/or scent preferences to the gustative and/or olfactive products.

### Generating a User Profile

A user profile can be created based on SNP genotyping and analyzing responses to the survey on personal characteristics and taste and/or scent preferences as described above. In addition, information on gustative and/or olfactive product preferences of a subject from sampling of typifying gustative and/or olfactive product may also be included. The user profile may be provided in a machine (e.g., a computer) readable format and/or in a hard (paper) copy.

In one embodiment, the user profile comprises information regarding a) the subject's genotype at one or more SNPs identified as useful for identifying taste and/or scent preference; b) information from a survey about the subject; c) the subject's taste and/or scent preference gustative and/or olfactive product scores for one or more gustative and/or olfactive products; and d) gustative and/or olfactive product bin scores for one or more gustative and/or olfactive product bins based on the genotyping from step (b), the information obtained about the subject from step (c), and the taste and/or scent preference scores for the one or more gustative and/or olfactive products from step (d), wherein the gustative and/or olfactive product bin scores are used to predict that the subject will prefer gustative and/or olfactive products from a gustative and/or olfactive product bin having a higher gustative and/or olfactive product bin score than a gustative and/or olfactive product bin having a lower gustative and/or olfactive product bin score.

The relationship between gustative and/or olfactive product preferences and SNP genotyping and survey responses can be evaluated, for example, using hierarchical and k-means clustering, consensus clustering, or ordered logistic regression to provide a gustative and/or olfactive product score for each of the typifying gustative and/or olfactive products. The gustative and/or olfactive product scores of the typifying gustative and/or olfactive products are then used to rank the gustative and/or olfactive product categories (i.e., gustative and/or olfactive product bins) to which the typifying gustative and/or olfactive product belong. Rankings can be represented by a gustative and/or olfactive product bin score, wherein increasing gustative and/or olfactive product bin scores are correlated with increasing preference for a category of gustative and/or olfactive product (i.e., a particular gustative and/or olfactive product bin).

Thus, subjects are given a “gustative and/or olfactive product score” for each typifying gustative and/or olfactive product indicating the degree of preference among the typifying gustative and/or olfactive products, and then given a “gustative and/or olfactive product bin score” for each gustative and/or olfactive product bin in the set of gustative and/or olfactive product bins corresponding to the broad categories of gustative and/or olfactive product to which the typifying gustative and/or olfactive products belong. The gustative and/or olfactive product bin scores are ranked, such that gustative and/or olfactive product bins that are likely to be preferred have higher scores than gustative and/or olfactive product bins that are likely to be less preferred or disliked.

A user taste and/or scent profile generated in this manner may be used to select gustative and/or olfactive products for an individual. Gustative and/or olfactive products are selected by ranking the gustative and/or olfactive product bins based on their scores, and offering the individual at least one gustative and/or olfactive product from the highest ranked gustative and/or olfactive product bin. If other gustative and/or olfactive product bins have positive rankings, that is, the individual has a higher than average preference for gustative and/or olfactive product from those gustative and/or olfactive product bins, then another gustative and/or olfactive product may be selected from another gustative and/or olfactive product bin having a positive ranking. The subject may be provided with one or more gustative and/or olfactive product samples from other gustative and/or olfactive product bins, preferably the top scoring gustative and/or olfactive product bins. For example, a subject may be provided with one or more gustative and/or olfactive product samples from one or more of the gustative and/or olfactive product bins having the top two, three, or four gustative and/or olfactive product bin scores. Gustative and/or olfactive product bins having negative rankings, indicating that the individual is unlikely to like the gustative and/or olfactive product, are not offered.

In certain embodiments, at least one gustative and/or olfactive product is provided to a subject based on the user taste and/or scent profile on a periodic basis. For example, gustative and/or olfactive products with positive rankings may be selected and shipped periodically as part of a gustative and/or olfactive product club.

System and Computerized Methods for Creating a Taste and/or Scent Profile

In a further aspect, the invention includes a computer implemented method for creating a taste and/or scent profile that predicts gustative and/or olfactive product preferences of a subject. The computer performs steps comprising: a) receiving inputted data comprising: i) genotyping information for the subject regarding which alleles are present at one or more SNPs identified as useful for identifying taste and/or scent preference, ii) values for the subject's taste and/or scent preference scores for a plurality of typifying gustative and/or olfactive products, and iii) information about the subject; b) calculating the subject's gustative and/or olfactive product bin scores for one or more gustative and/or olfactive product bins based on the subject's taste and/or scent preference scores for one or more gustative and/or olfactive products, wherein the gustative and/or olfactive product bin scores are used to predict that the subject will prefer gustative and/or olfactive products from a gustative and/or olfactive product bin having a higher gustative and/or olfactive product bin score than a gustative and/or olfactive product bin having a lower gustative and/or olfactive product bin score; and c) displaying information regarding predicted gustative and/or olfactive product preferences of the subject. In one embodiment, the inputted data comprises values for the subject's taste and/or scent preference scores for one or more gustative and/or olfactive products. In another embodiment, the computer implemented method further comprises storing a user profile for the subject comprising information regarding the subject's gustative and/or olfactive product preferences.

In certain embodiments, the computer implemented method further comprises inputting a list of gustative and/or olfactive products from a commercial establishment (e.g., restaurant, bar, winery, cosmetics retailer, or store), and displaying a listing of gustative and/or olfactive products available at the commercial establishment that belong to the gustative and/or olfactive product bin having the highest gustative and/or olfactive product bin score. The list of gustative and/or olfactive products from a commercial establishment may be obtained, for example, by providing an image of a menu from the commercial establishment, and processing the image to obtain the list of gustative and/or olfactive products. Alternatively, the list of gustative and/or olfactive products may be obtained by identifying the commercial establishment, and retrieving an electronic representation of the list of gustative and/or olfactive products from a gustative and/or olfactive products list database. In certain embodiments, the computer implemented method further comprises displaying a listing of gustative and/or olfactive products available at a commercial establishment that having positive rankings based on their gustative and/or olfactive product bin scores. The gustative and/or olfactive products from different gustative and/or olfactive product bins may be displayed in the order of their ranking and the display may be color coded to differentiate gustative and/or olfactive products from different gustative and/or olfactive product bins. The display may be adjustable to allow control over how gustative and/or olfactive products are listed. In certain embodiments, the display can be adjusted to list only certain gustative and/or olfactive products with positive rankings that are available at a commercial establishment, such as only gustative and/or olfactive products that belong to the gustative and/or olfactive product bin having the top gustative and/or olfactive product bin score, or gustative and/or olfactive products that belong to gustative and/or olfactive product bins having the top two, three, or four gustative and/or olfactive product bin scores, or any other desired listing.

In a further aspect, the invention includes a system for performing the computer implemented method, as described. The system may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions for creating a taste and/or scent profile that predicts gustative and/or olfactive product preferences of a subject. For example, the storage component includes instructions for calculating taste and/or scent preference scores for typifying gustative and/or olfactive products and gustative and/or olfactive product bin scores for ranking gustative and/or olfactive products according to predicted preferences of a subject, as described herein (e.g., see Examples). The computer processor is coupled to the storage component and configured to execute the instructions stored in the storage component in order to receive data regarding the subject and analyze data according to one or more algorithms. The display component displays information regarding the predicted gustative and/or olfactive products preferences of the subject.

The storage component may be of any type capable of storing information accessible by the processor, such as a hard-drive, memory card, ROM, RAM, DVD, CD-ROM, USB Flash drive, write-capable, and read-only memories. The processor may be any well-known processor, such as processors from Intel Corporation. Alternatively, the processor may be a dedicated controller such as an ASIC.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the diagnostic system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

In certain embodiments, the processor and storage component may comprise multiple processors and storage components that may or may not be stored within the same physical housing. For example, some of the instructions and data may be stored on a removable DVD and others within a read-only computer chip. Some or all of the instructions and data may be stored in a location physically remote from, yet still accessible by, the processor. Similarly, the processor may actually comprise a collection of processors which may or may not operate in parallel.

In one aspect, the computer is a server communicating with one or more client computers. Each client computer may be configured similarly to the server, with a processor, storage component and instructions. Each client computer may be a personal computer, intended for use by a person, having all the internal components normally found in a personal computer such as a central processing unit (CPU), display (for example, a monitor displaying information processed by the processor), DVD, hard-drive, user input device (for example, a mouse, keyboard, touch-screen or microphone), speakers, modem and/or network interface device (telephone, cable or otherwise) and all of the components used for connecting these elements to one another and permitting them to communicate (directly or indirectly) with one another. Moreover, computers in accordance with the systems and methods described herein may comprise any device capable of processing instructions and transmitting data to and from humans and other computers including network computers lacking local storage capability.

Although the client computers may comprise a full-sized personal computer, many aspects of the system and method are particularly advantageous when used in connection with mobile devices capable of wirelessly exchanging data with a server over a network such as the Internet. For example, client computer may be a wireless-enabled PDA such as a Blackberry phone, Apple iPhone, Android phone, or other Internet-capable cellular phone. In such regard, the user may input information using a small keyboard, a keypad, a touch screen, or any other means of user input. The computer may have an antenna for receiving a wireless signal.

The server and client computers are capable of direct and indirect communication, such as over a network. It should be appreciated that a typical system can include a large number of connected computers, with each different computer being at a different node of the network. The network, and intervening nodes, may comprise various combinations of devices and communication protocols including the Internet, World Wide Web, intranets, virtual private networks, wide area networks, local networks, cell phone networks, private networks using communication protocols proprietary to one or more companies, Ethernet, WiFi and HTTP. Such communication may be facilitated by any device capable of transmitting data to and from other computers, such as modems (e.g., dial-up or cable), networks and wireless interfaces. The server may be a web server.

Although certain advantages are obtained when information is transmitted or received as noted above, other aspects of the system and method are not limited to any particular manner of transmission of information. For example, in some aspects, information may be sent via a medium such as a disk, tape, flash drive, memory card, DVD, or CD-ROM. In other aspects, the information may be transmitted in a non-electronic format and manually entered into the system. Yet further, although some functions are indicated as taking place on a server and others on a client, various aspects of the system and method may be implemented by a single computer having a single processor.

### Kits

Kits for performing the desired method(s) are also provided, and comprise a container or housing for holding the components of the kit, one or more vessels containing one or more nucleic acid(s), and optionally one or more vessels containing one or more reagents. Any composition described herein may be included in the kit, including those reagents useful for performing the methods described, including reagents for SNP genotyping, such as one or more allele specific-probes, primers or primer pairs, enzymes (including polymerases and ligases), intercalating dyes, labeled probes, and labels that can be incorporated into amplification products.

In some embodiments, the kit comprises allele-specific probes and/or primers or primer pairs for determining which alleles are present at one or more SNPs identified as useful for identifying taste and/or scent preference. The kit may include allele-specific primers or pairs of primers suitable for selectively amplifying the target sequences of a SNP allele. The kit may comprise at least 2, 3, 4, 5, 6, 7, 8, 9, 10, or more allele-specific primers or pairs of primers suitable for selectively amplifying one or more target SNP alleles.

In some embodiments, the primers or primer pairs of the kit, when used in an amplification reaction, specifically amplify a non-coding target, coding target, exonic, or non-exonic target described herein, a nucleic acid sequence corresponding to a SNP allele identified as useful for identifying taste and/or scent preference, an RNA form thereof, or a complement to either thereof. The kit may include a plurality of such primers or primer pairs which can specifically amplify a corresponding plurality of different target sequence, including a non-coding target, coding target, exonic, or non-exonic transcript described herein, a nucleic acid sequence corresponding to a target selected identified as useful for identifying taste and/or scent preference, RNA forms thereof, or complements thereto.

The reagents may independently be in liquid or solid form. The reagents may be provided in mixtures. Control samples and/or nucleic acids may optionally be provided in the kit.

The nucleic acids may be provided in an array format, and thus a SNP array or microarray may be included in the kit. For example, the kit may include a SNP array comprising a set of allele-specific probes for detecting which alleles are present at one or more SNPs identified as useful for identifying taste and/or scent preference.

Instructions for using the kit to perform one or more methods of the invention can be provided with the container, and can be provided in any fixed medium. The instructions may be located inside or outside the container or housing, and/or may be printed on the interior or exterior of any surface thereof. A kit may be in multiplex form for concurrently detecting and/or quantitating one or more different target polynucleotides representing the expressed target sequences.

### Data Analysis

In some embodiments, one or more pattern recognition methods can be used in analyzing SNP genotyping data and information regarding a subject's personal characteristics and taste and/or scent preferences, such as determined from querying the subject and/or gustative and/or olfactive product sampling as described herein. Developing models that predict individual gustative and/or olfactive product preferences may comprise the use of a machine learning algorithm. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLES

### Example 1: Genetic Profiling to Predict Individual Taste Preferences and Wine Selection Based on Genetic Profiles

Genetic profiling to predict individual wine taste preferences and selection of wine based on genetic profiles was performed as follows. Adult participants were surveyed concerning their taste preferences, eating and drinking habits, and relevant demographic questions. Additionally, they were offered twelve wines encompassing a number of grape varietals, and questioned on their preferences among these wines and their ability to detect defined flavors in them. A total of 578 questions were asked. The surveys were conducted prior to and at nine different wine tasting events, with 541 people surveyed in total. SurveyMonkey.com was used to digitally collect survey responses from participants. Informed consent for these results to be used for research purposes was obtained.

Subsequently forty-one (41) single nucleotide variants (SNPs) were assayed on all survey participants (Table 1). Variants were chosen by literature review to identify a variety of alleles with likely association with differences in the ability to perceive certain tastes. Saliva was collected by survey participants using a kit supplied by DNA Genotek (Ottawa, Canada) and genomic DNA isolated using the NA Genotek PrepIT*L2P saliva protocol as per manufacturers recommendation. Genotyping was performed by the HudsonAlpha Institute for Biotechnology (Huntsville, Ala.). Briefly, genomic DNA quantity was measured using Invitrogen's PicoGreen assay. Taqman custom and prepared SNP assays were obtained from Applied Biosystems/Thermo. Assays were provided as a probe/primer mix, lyophilized in a 384-well plate. Assays were plated by Thermo in duplicate, with four sets of assays loaded per plate. Genomic DNA samples were mixed with an equivalent amount of 2×master mix in a sterile reservoir, and the mixture was then aliquoted with a Biomek FxP robot to the 384-well plates. The final reaction volume of each well was 5 PCR reactions were run using manufacturer recommended conditions on an ABI thermal cycler (40 cycles). Completed reactions were analyzed using the Applied Biosystems QuantStudio 12K flex system.

TABLE 1
Genetic Variants (SNPs) for Taste Preference

| | | | Major | Minor | | Reference (include | | | Association with |
|---|---|---|---|---|---|---|---|---|---|
| | | | allele | allele | PubMed | first author, title, | | Primary | primary |
| rsId | Chr | Pos | dbSNP | dbSNP | ID | journal, & date) | Gene | phenotype | phenotype |
| rs838133 | 19 | 49259529 | G | A | NA | 23 and Me White | FGF21 intron | Sweet: | Higher odds of |
| | | | | | | Paper 23-08, | | preference | preferring sweet- |
| | | | | | | Genetic associations | | | tasting foods over |
| | | | | | | with traits in | | | salty/savory |
| | | | | | | 23 and Me customers | | | |
| rs1421085 | 16 | 53800954 | T | C | NA | 23 and Me White | FTO intron | Sweet: | Higher odds of |
| | | | | | | Paper 23-08, | | preference | preferring sweet- |
| | | | | | | Genetic associations | | | tasting foods over |
| | | | | | | with traits in | | | salty/savory |
| | | | | | | 23 and Me customers | | | |
| rs3930459 | 11 | 7953958 | T | C | NA | 23 and Me White | non-coding | Bitter: | Higher odds of |
| | | | | | | Paper 23-08, | regions within | Cilantro | disliking cilantro |
| | | | | | | Genetic associations | cluster of 56 | preference | |
| | | | | | | with traits in | OR genes, | | |
| | | | | | | 23 and Me customers | within 10 kb | | |
| | | | | | | | OR10A2, | | |
| | | | | | | | OR10A4, | | |
| | | | | | | | OR10A6, and | | |
| | | | | | | | OR10A3 and | | |
| | | | | | | | within 100 kb | | |
| | | | | | | | of OR6A2 | | |
| | | | | | | | (Xsm 11 OR | | |
| | | | | | | | cluster 1) | | |
| rs2274333 | 1 | 9017204 | A | G | 21712049 | Calo, C., Padiglia, A., | CA6 (gustin) | Bitter: | Higher bitter taste |
| | | | | | | Zonza, A., | | perception | perception, Gustin |
| | | | | | | Corrias, L., Contu, P., | | | modulates |
| | | | | | | Tepper, B. J., et | | | sensitivity to PROP |
| | | | | | | al. (2011). | | | |
| | | | | | | Polymorphisms in | | | |
| | | | | | | TAS2R38 and the | | | |
| | | | | | | taste bud trophic | | | |
| | | | | | | factor, gustin gene | | | |
| | | | | | | cooperate | | | |
| | | | | | | in modulating PROP | | | |
| | | | | | | taste phenotype. | | | |
| | | | | | | Physiology & | | | |
| | | | | | | Behavior, 104(5), | | | |
| | | | | | | 1065-1071. | | | |
| rs111615792 | 1 | 1267651 | G | A | 19587085 | Chen Q Y, Alarcon S, | TAS1R3 | Umami: | Increased umami |
| | | | | | | Tharp A, Ahmed O M, | | perception | taste perception |
| | | | | | | Estrella N L, | | | (doubling of umami |
| | | | | | | Greene T A, Rucker J, | | | ratings of 200 mmol |
| | | | | | | Breslin P A. 2009. | | | MPG/L) |
| | | | | | | Perceptual variation | | | |
| | | | | | | in umami taste and | | | |
| | | | | | | polymorphismsin | | | |
| | | | | | | TAS1R taste | | | |
| | | | | | | receptor genes. Am | | | |
| | | | | | | J Clin Nutr. | | | |
| | | | | | | 90: 770S-779S. | | | |
| rs72921001 | 11 | 6889648 | C | A | NA | Eriksson N, et al. | w/in 100 kb of | Bitter: | soapy taste |
| | | | | | | 2012. A genetic | OR6A2 | Cilantro | detection |
| | | | | | | variant near olfactory | | preference | |
| | | | | | | receptor genes | | (soapy | |
| | | | | | | influences cilantro | | taste) | |
| | | | | | | preference. Flavour | | | |
| | | | | | | 1: 22. | | | |
| rs7792845 | 7 | 80151369 | C | T | 20660057 | Fushan, A. A., | GNAT3 | Sweet: | Increased sucrose |
| | | | | | | Simons, C. T., Slack, J. P., | (gustducin) | sucrose | discrimination |
| | | | | | | & Drayna, D. | | sensitivity | |
| | | | | | | (2010). Association | | | |
| | | | | | | between | | | |
| | | | | | | common variation in | | | |
| | | | | | | genes encoding | | | |
| | | | | | | sweet taste signaling | | | |
| | | | | | | components and | | | |
| | | | | | | human sucrose | | | |
| | | | | | | perception. | | | |
| | | | | | | Chemical Senses, | | | |
| | | | | | | 35(7), 579-592. | | | |
| rs10772420 | 12 | 11174276 | G | A | 21163912 | Hayes, et al. 2011. | TAS2R19 | Bitter: | Less bitterness |
| | | | | | | Allelic variation in | | Grapefruit | perception and |
| | | | | | | TAS2R bitter | | juice | higher liking of |
| | | | | | | receptor genes | | | grapefruit juice |
| | | | | | | associates with | | | |
| | | | | | | variation in | | | |
| | | | | | | sensations from and | | | |
| | | | | | | ingestive behaviors | | | |
| | | | | | | toward common | | | |
| | | | | | | bitter beverages in | | | |
| | | | | | | adults. Chem | | | |
| | | | | | | Senses 36: 311. | | | |
| rs846672 | 7 | 122630180 | C | A | 21163912 | Hayes, et al. 2011. | TAS2R16 | Alcohol | AA consumed |
| | | | | | | Allelic variation in | | trends: | alcohol twice as |
| | | | | | | TAS2R bitter | | consumption | frequently as |
| | | | | | | receptor genes | | | heterozygotes or |
| | | | | | | associates with | | | major allele |
| | | | | | | variation in | | | homozygotes, AA |
| | | | | | | sensations from and | | | also consumed |
| | | | | | | ingestive behaviors | | | larger amounts. |
| | | | | | | toward common | | | |
| | | | | | | bitter beverages in | | | |
| | | | | | | adults. Chem | | | |
| | | | | | | Senses 36: 311. | | | |
| rs9295791 | 6 | 29096033 | A | G | NA | Jaeger, et al. 2010. | OR2J3 (near) | Odor: cis-3-hexen-1-ol (grassy | |
| | | | | | | A preliminary | | odor) detection | |
| | | | | | | investigation into a | | | |
| | | | | | | genetic | | | |
| | | | | | | basis for cis-3- | | | |
| | | | | | | hexen-1-ol odour | | | |
| | | | | | | perception: a | | | |
| | | | | | | genome wide | | | |
| | | | | | | assocation | | | |
| | | | | | | approach. Food | | | |
| | | | | | | Qual Pref 21: 121 | | | |
| rs6591536 | 11 | 59211188 | A | G | 23910657 | Jaeger, et al. 2013. | OR5A1 | Odor: b- | greater sensitivity |
| | | | | | | A Mendelian trait for | | ionone | to |
| | | | | | | olfactory sensitivity | | sensitivity | b-ionone |
| | | | | | | affects odor | | | |
| | | | | | | experience and food | | | |
| | | | | | | selection. Curr Biol | | | |
| | | | | | | 23: 1601. | | | |
| rs1761667 | 7 | 80244939 | G | A | 22240721 | Keller, et al. 2012. | CD36 | Other: Fat | Salad dressings |
| | | | | | | Common variants in | | perception | tasted creamier |
| | | | | | | the CD36 | | | |
| | | | | | | gene are associated | | | |
| | | | | | | with oral fat | | | |
| | | | | | | perception, fat | | | |
| | | | | | | preferences, and | | | |
| | | | | | | obesity | | | |
| | | | | | | in African Americans | | | |
| rs10772397 | 12 | 11138683 | T | C | 22977065 | Knaapila, et al. | TAS2R50 | Bitter: Cilantro pleasantness | |
| | | | | | | 2012. Genetic | | | |
| | | | | | | analysis of | | | |
| | | | | | | chemosensory traits | | | |
| | | | | | | in human twins. | | | |
| | | | | | | Chem Senses | | | |
| | | | | | | 37: 869. | | | |
| rs586965 | 1 | 1226348 | G | C | 22977065 | Knaapila, et al. | SCNN1D | Alcohol trends: Ethanol burn | |
| | | | | | | 2012. Genetic | (salt receptor | | |
| | | | | | | analysis of | gene) | | |
| | | | | | | chemosensory traits | | | |
| | | | | | | in human twins. | | | |
| | | | | | | Chem Senses | | | |
| | | | | | | 37: 869. | | | |
| rs5020278 | 19 | 9325116 | G | A | 22977065 | Knaapila, et al. | ORD74 | Alcohol | Ethanol burn |
| | | | | | | 2012. Genetic | | trends: | |
| | | | | | | analysis of | | Ethanol | |
| | | | | | | chemosensory traits | | burn | |
| | | | | | | in human twins. | | | |
| | | | | | | Chem Senses | | | |
| | | | | | | 37: 869. | | | |
| rs11988795 | 8 | 72949601 | C | T | 22977065 | Knaapila, et al. | TRPA1 | Bitter: Cilantro pleasantness | |
| | | | | | | 2012. Genetic | | | |
| | | | | | | analysis of | | | |
| | | | | | | chemosensory traits | | | |
| | | | | | | in human twins. | | | |
| | | | | | | Chem Senses | | | |
| | | | | | | 37: 869. | | | |
| rs4595035 | 7 | 143141475 | C | T | 22977065 | Knaapila, et al. | TAS2R60 | Bitter: Basil pleasantness | |
| | | | | | | 2012. Genetic | | | |
| | | | | | | analysis of | | | |
| | | | | | | chemosensory traits | | | |
| | | | | | | in human twins. | | | |
| | | | | | | Chem Senses | | | |
| | | | | | | 37: 869. | | | |
| rs12226920 | 12 | 11150046 | G | T | 22977065 | Knaapila, et al. | TAS2R20 | Bitter: | |
| | | | | | | 2012. Genetic | | Quinine | |
| | | | | | | analysis of | | | |
| | | | | | | chemosensory traits | | | |
| | | | | | | in human twins. | | | |
| | | | | | | Chem Senses | | | |
| | | | | | | 37: 869. | | | |
| rs1524600 | 7 | 80138303 | G | A | 22977065 | Knaapila, et al. | GNAT3 | Bitter: Cilantro pleasantness | |
| | | | | | | 2012. Genetic | | | |
| | | | | | | analysis of | | | |
| | | | | | | chemosensory traits | | | |
| | | | | | | in human twins. | | | |
| | | | | | | Chem Senses | | | |
| | | | | | | 37: 869. | | | |
| rs61729907 | 19 | 9325252 | G | A | 22977065 | Knaapila, et al. | ORD74 | Alcohol | Ethanol burn |
| | | | | | | 2012. Genetic | | trends: | |
| | | | | | | analysis of | | Ethanol | |
| | | | | | | chemosensory traits | | burn | |
| | | | | | | in human twins. | | | |
| | | | | | | Chem Senses | | | |
| | | | | | | 37: 869. | | | |
| rs1548803 | 12 | 10959031 | T | C | 22977065 | Knaapila, et al. | TAS2R38 | Bitter: | |
| | | | | | | 2012. Genetic | | Quinine | |
| | | | | | | analysis of | | | |
| | | | | | | chemosensory traits | | | |
| | | | | | | in human twins. | | | |
| | | | | | | Chem Senses | | | |
| | | | | | | 37: 869. | | | |
| rs4684677 | 3 | 10328453 | T | A | 21448464 | Landgren, et al. | GHRL | Sweet: Sucrose intake and | |
| | | | | | | 2011. PLos One. | | alcohol consumption | |
| | | | | | | 6: e18170 | | | |
| rs42451 | 3 | 10330377 | C | T | 21448465 | Landgren, et al. | GHRL | Sweet: Sucrose intake and | |
| | | | | | | 2011. PLos One. | | alcohol consumption | |
| | | | | | | 6: e18171 | | | |
| rs35680 | 3 | 10330564 | T | C | 21448466 | Landgren, et al. | GHRL | Sweet: Sucrose intake and | |
| | | | | | | 2011. PLos One. | | alcohol consumption | |
| | | | | | | 6: e18172 | | | |
| rs34911341 | 3 | 10331519 | C | T | 21448467 | Landgren, et al. | GHRL | Sweet: Sucrose intake and | |
| | | | | | | 2011. PLos One. | | alcohol consumption | |
| | | | | | | 6: e18173 | | | |
| rs696217 | 3 | 10331457 | G | T | 21448468 | Landgren, et al. | GHRL | Sweet: Sucrose intake and | |
| | | | | | | 2011. PLos One. | | alcohol consumption | |
| | | | | | | 6: e18174 | | | |
| rs26802 | 3 | 10332365 | T | G | 21448469 | Landgren, et al. | GHRL | Sweet: Sucrose intake and | |
| | | | | | | 2011. PLos One, | | alcohol consumption | |
| | | | | | | 6: e18175 | | | |
| rs2708377 | 12 | 11216315 | T | C | 23966204 | Ledda, et al. 2014. | TAS2R46 | Bitter: | Less intense |
| | | | | | | GWAS of human bitter | | Caffeine/ | bitterness |
| | | | | | | taste perception | | Coffee | perception |
| | | | | | | identifies new | | | |
| | | | | | | loci and reveals | | | |
| | | | | | | additional complexity | | | |
| | | | | | | of bitter taste | | | |
| | | | | | | genetics | | | |
| rs28757581 | 6 | 29080004 | A | G | 22714804 | McRae, et al. 2012. | OR2J3 | Odor: cis-3-hexen-1-ol (grassy | |
| | | | | | | Genetic variation in | | odor) detection | |
| | | | | | | the odorant receptor | | | |
| | | | | | | OR2J3 is associated | | | |
| | | | | | | with the ability to | | | |
| | | | | | | detect the “grassy” | | | |
| | | | | | | smelling odor, cis-3- | | | |
| | | | | | | hexen-1-ol | | | |
| rs35744813 | 1 | 1265460 | C | T | 22546773 | Mennella, et al. | TAS1R3 | Sweet: | Reduced ability to |
| | | | | | | 2012. The proof is in | | Sucrose | detect sucrose, |
| | | | | | | the pudding: children | | preference | therefore increased |
| | | | | | | prefer lower fat but | | | sucrose preference |
| | | | | | | higher sugar than do | | | |
| | | | | | | mothers. | | | |
| rs4920566 | 1 | 19179824 | G | A | 22888812 | Piratsu, et al. 2012. | TAS1R2 | Alcohol | White wine liking |
| | | | | | | Genetics of food | | trends: | (P = 4.0 × 10−4) |
| | | | | | | preferences: a first | | Wine liking | |
| | | | | | | view from silk road | | | |
| | | | | | | populations. J Food | | | |
| | | | | | | Sci, 77: S413. | | | |
| rs3935570 | 1 | 19167371 | G | T | 22888812 | Piratsu, et al. 2012. | TAS1R2 | Alcohol | White wine liking |
| | | | | | | Genetics of food | | trends: | (P = 4.0 × 10−4) |
| | | | | | | preferences: a first | | Wine liking | |
| | | | | | | view from silk road | | | |
| | | | | | | populations. J Food | | | |
| | | | | | | Sci, 77: S413. | | | |
| rs2277675 | 17 | 3500510 | T | C | 22888812 | Piratsu, et al. 2012. | TRPV1 | Beet liking | |
| | | | | | | Genetics of food | | | |
| | | | | | | preferences: a first | | | |
| | | | | | | view from silk road | | | |
| | | | | | | populations. J Food | | | |
| | | | | | | Sci, 77: S413. | | | |
| rs71443637 | 12 | 11244194 | C | T | 24647340 | Piratsu, et al. 2014. | TAS2R43 | Bitter: | Increased coffee |
| | | | | | | Association Analysis | | Caffeine/ | liking (close to |
| | | | | | | of Bitter Receptor | | Coffee | significance) |
| | | | | | | Genes in Five | | | |
| | | | | | | Isolated Populations | | | |
| | | | | | | Identifies a | | | |
| | | | | | | Significant | | | |
| | | | | | | Correlation | | | |
| | | | | | | between TAS2R43 | | | |
| | | | | | | Variants and Coffee | | | |
| | | | | | | Liking | | | |
| rs9276975 | 6 | 32973599 | C | T | 25758996 | Piratsu, et al. 2015. | HLA-DOA | Alcohol | White wine liking |
| | | | | | | Genome-wide | | trends: | (p = 2.1 × 10−8), |
| | | | | | | association analysis | | Wine liking | Red wine liking |
| | | | | | | on five isolated | | | (p = 8.3 × 10−6) |
| | | | | | | populations identifies | | | |
| | | | | | | variants of the HLA- | | | |
| | | | | | | DOA gene | | | |
| | | | | | | associated with | | | |
| | | | | | | white wine liking. Eur | | | |
| | | | | | | J Hum Genet Pub | | | |
| | | | | | | online Mar. 11, | | | |
| | | | | | | 2015 | | | |
| rs34160967 | 1 | 6635306 | G | A | 19696921 | Shigemura, et al. | TAS1R1 | Umami: | more sensitive |
| | | | | | | 2009. PLoS One. | | perception | unami receptor |
| | | | | | | 4: e6717. | | | |
| rs307377 | 1 | 1269554 | C | T | 19696921 | Shigemura, et al. | TAS1R3 | Umami: | more sensitive |
| | | | | | | 2009. PLoS One. | | perception | unami receptor |
| | | | | | | 4: e6717. | | | |
| rs713598 | 7 | 141673345 | G | C | 17250611 | Wang, et al. 2007. | TAS2R38 | Alcohol | Higher mean |
| | | | | | | Alcoholism: Clinical | | trends: | Maxdrinks scores |
| | | | | | | and Experimental | | consumption | in AA but not EA |
| | | | | | | Research. 31: 209. | | | |
| rs1726866 | 7 | 141672705 | G | A | 17250611 | Wang, et al. 2007. | TAS2R38 | Alcohol | Higher mean |
| | | | | | | Alcoholism: Clinical | | trends: | Maxdrinks scores |
| | | | | | | and Experimental | | consumption | in AA but not EA |
| | | | | | | Research. 31: 209. | | | |
| rs10246939 | 7 | 141672604 | C | T | 17250611 | Wang, et al. 2007. | TAS2R38 | Alcohol | Higher mean |
| | | | | | | Alcoholism: Clinical | | trends: | Maxdrinks scores |
| | | | | | | and Experimental | | consumption | in AA but not EA |
| | | | | | | Research. 31: 209. | | | |
| rs846664 | 7 | 122635173 | G | T | 16051168 | Soranzo, et al. 2005. | TAS2R16 | Bitter: | Increased |
| | | | | | | Positive selection on | | Sensitivity | sensitivity to |
| | | | | | | a high-sensitivity | | to salicin, | salicin, arbutin, and |
| | | | | | | allele of the human | | arbutin, and | amygdalin |
| | | | | | | bitter taste receptor | | amygdalin | |
| | | | | | | TAS2R16 | | | |

Survey data was encoded numerically, with taste preferences encoded as −1 to +1, representing “dislike”, “no preference”, and “like”. Other questions were similarly encoded. Genotypes were encoded as 0, 1, or 2 for the minor homozygous, heterozygous, and major homozygous alleles, respectively. Participants were randomly assigned to a discovery and validation cohort, with ⅔ of participants being in the discovery cohort, and ⅓ in the validation cohort.

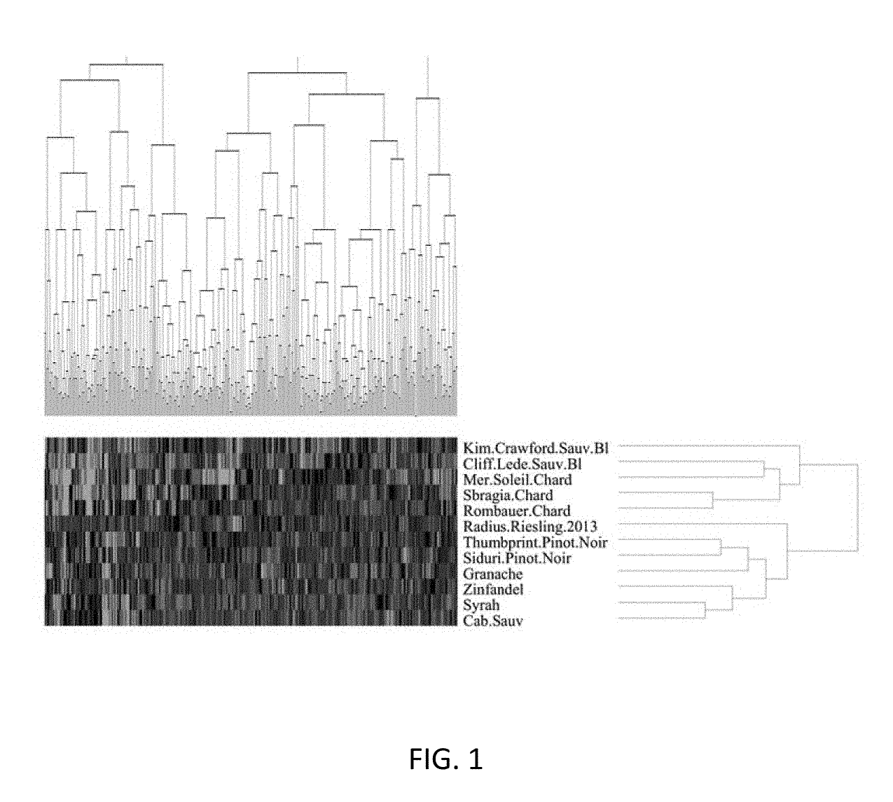

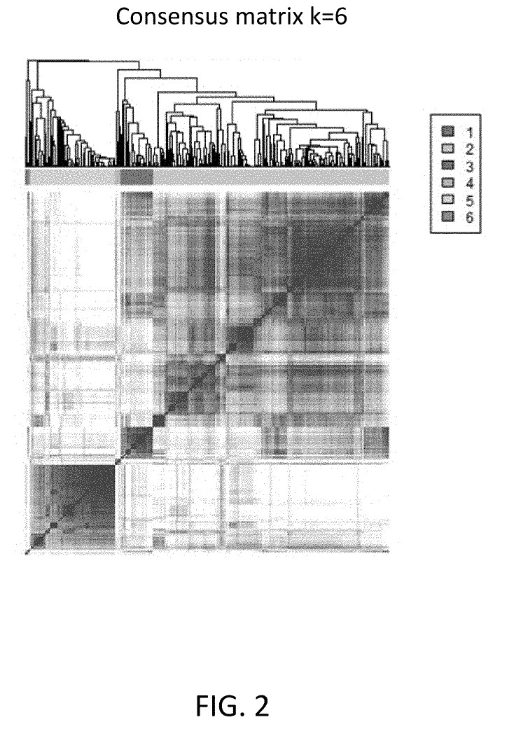

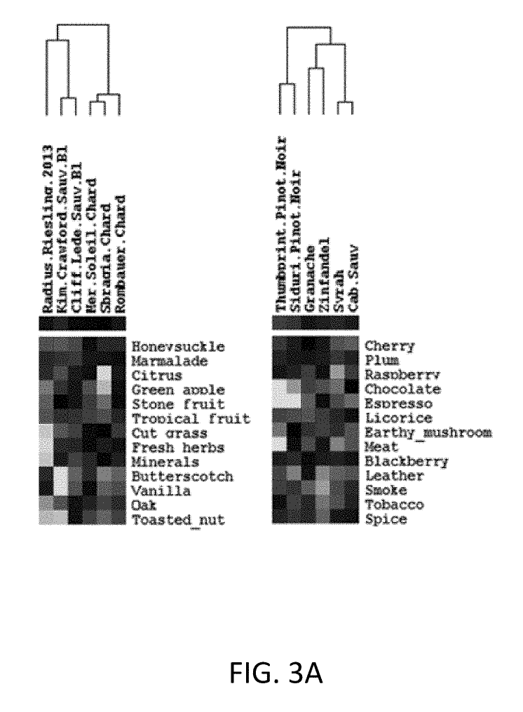

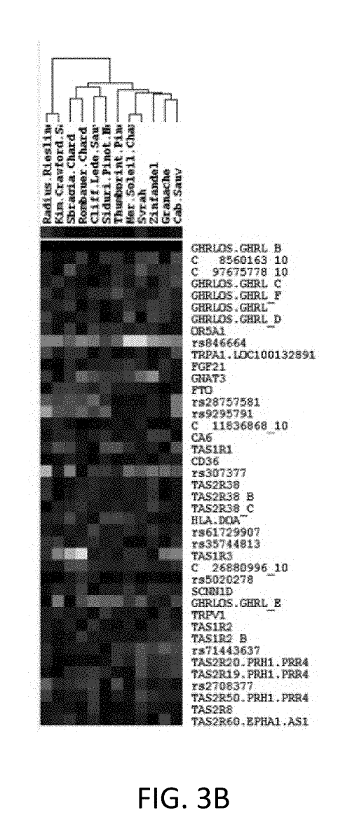

Initial exploration of the relationship between wine preferences was assessed using hierarchical and k-means clustering (FIG. 1). Relationships between wine preferences was also gauged using consensus clustering (FIG. 2), which allowed the determination of a reduced number of dimensions which could be used to describe wine preference. Association between wine preferences and survey responses and genotype was also explored using hierarchical clustering (FIGS. 3A, 3B) and assessed using ordered logistic regression.

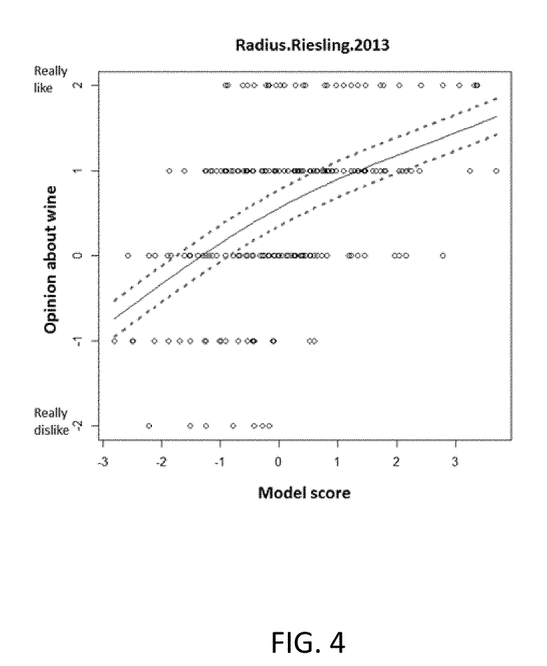

Variables for models predicting preference for the twelve wines were chosen from those survey responses and genetic variants having the greatest effect size (with any of the wines) and for which the 95% confidence intervals were both positive or both negative. Elastic net regression is used to remove variables not significantly independent of other variables. For the final model, thirteen survey responses and 5 genetic variants (Table 2) were selected and used to model each of the twelve wines (FIG. 4). Using the same variable for each typifying wine allows the score given to each to be comparable to the other wines. The effect size from the ordered logistic regression was used as the coefficient for the variables in the wine preference models.

TABLE 2
Coefficients Associated with Model Questions and Variants

| | Sweet | Taste | Taste | | Wine | Drinks | | | Wine | | Taste | | Taste | | | | | |
|---|---|---|---|---|---|---|---|---|---|---|---|---|---|---|---|---|---|---|
| | vs. | Pref. | Pref. | Gender | Knowledge | Wine | Taste | Taste | Knowl- | Taste | Pref. | Cheese | Pref. | | | | | |
| | Oak | Mush- | Black | Re- | Tries | Freq. | Pref. | Pref. | edge | Pref. | Sweet | Pref. | Black- | | | | | |
| | Response | rooms | Coffee | sponse | New | Response | Savory | Grass | Learning | Peaches | Coffee | Swiss | berries | rs3930459 | rs42451 | rs2708377 | rs71443637 | rs12226920 |
| Radius | −0.7070 | −0.3674 | −0.0040 | 0.2200 | 0.0610 | −0.5627 | 0.6159 | 0.2581 | 0.8209 | 0.9655 | 0.3143 | 0.377649 | −0.13437 | 0.00 | 0.00 | 0.00 | 0.00 | 0.00 |
| Riesling | | | | | | | | | | | | | | | | | | |
| 2013 | | | | | | | | | | | | | | | | | | |
| Kim | 0.1598 | 0.5625 | 0.2319 | −0.0846 | 0.4667 | 0.2691 | 0.5467 | 0.2350 | 0.1694 | 0.2174 | −0.1746 | 0.405738 | 0.244817 | 0.00 | 0.00 | 0.00 | 0.00 | 0.00 |
| Crawford | | | | | | | | | | | | | | | | | | |
| Sauv | | | | | | | | | | | | | | | | | | |
| Bl | | | | | | | | | | | | | | | | | | |
| Cliff | 0.3616 | 0.4960 | 0.2617 | −0.5147 | 0.2776 | 0.6326 | 0.3980 | 0.3966 | −0.3697 | 0.0507 | −0.2990 | −0.06705 | −0.04484 | 0.00 | 0.00 | 0.00 | 0.00 | 0.00 |
| Lede | | | | | | | | | | | | | | | | | | |
| Sauv | | | | | | | | | | | | | | | | | | |
| Bl | | | | | | | | | | | | | | | | | | |
| Mer | 0.3621 | 0.1043 | 0.2325 | −0.6683 | −0.0689 | 0.1524 | 0.1445 | 0.1194 | 0.0514 | 0.1733 | −0.1551 | 0.285404 | 0.157368 | 0.00 | 0.45 | 0.00 | 0.00 | 0.00 |
| Soleil | | | | | | | | | | | | | | | | | | |
| Chard | | | | | | | | | | | | | | | | | | |
| Sbragia | 0.7345 | 0.1975 | 0.1921 | −0.3180 | 0.2836 | 0.2816 | −0.1413 | 0.1266 | −0.0027 | −0.3447 | −0.1186 | 0.498293 | 0.280204 | 0.40 | 0.00 | 0.00 | 0.00 | 0.00 |
| Chard | | | | | | | | | | | | | | | | | | |
| Rombauer | 0.7313 | 0.4120 | 0.2476 | −0.5965 | 0.1898 | 0.3372 | 0.1399 | 0.1094 | 0.1285 | −0.1260 | −0.1122 | 0.314874 | 0.059932 | 0.43 | 0.00 | 0.00 | 0.00 | 0.00 |
| Chard | | | | | | | | | | | | | | | | | | |
| Thumbprint | 0.6101 | 0.3863 | 0.3890 | −0.4984 | 0.5692 | 0.5367 | 0.7187 | 0.1918 | −0.2174 | 0.1889 | 0.0729 | −0.13314 | 0.688492 | 0.00 | 0.00 | 0.40 | 0.00 | −0.28 |
| Pinot | | | | | | | | | | | | | | | | | | |
| Noir | | | | | | | | | | | | | | | | | | |
| Siduri | 0.4511 | 0.2911 | 0.4865 | −0.3183 | 0.8014 | 0.2144 | 0.3938 | 0.2427 | 0.2594 | 0.0824 | −0.2415 | 0.429498 | 0.65768 | 0.00 | 0.00 | 0.00 | 0.00 | 0.00 |
| Pinot | | | | | | | | | | | | | | | | | | |
| Noir | | | | | | | | | | | | | | | | | | |
| Grenache | 0.7502 | 0.0817 | 0.2614 | −0.0860 | 0.6897 | 0.4433 | 0.2780 | 0.2058 | −0.4170 | 0.1805 | −0.0464 | −0.447 | 0.783813 | 0.00 | 0.00 | 0.00 | 0.49 | −0.27 |
| Zinfandel | 0.6424 | 0.0344 | 0.3501 | −0.1499 | 0.6028 | 0.1956 | 0.3077 | −0.0243 | −0.2566 | −0.2748 | −0.3745 | −0.6555 | 0.322897 | 0.00 | 0.00 | 0.00 | 0.34 | 0.00 |
| Syrah | 0.8241 | 0.1071 | 0.4049 | −0.3238 | 0.5602 | 0.6074 | 0.1244 | −0.0673 | 0.0730 | 0.1045 | −0.3109 | −0.45071 | 0.875547 | 0.00 | 0.00 | 0.00 | 0.43 | −0.41 |
| Cab | 0.7984 | 0.1887 | 0.7024 | −0.6284 | 0.3956 | 0.5565 | 0.4108 | 0.2052 | −0.1702 | −0.0939 | −0.3482 | −0.36389 | 0.973206 | 0.00 | 0.00 | 0.00 | 0.37 | −0.33 |
| Sauv | | | | | | | | | | | | | | | | | | |

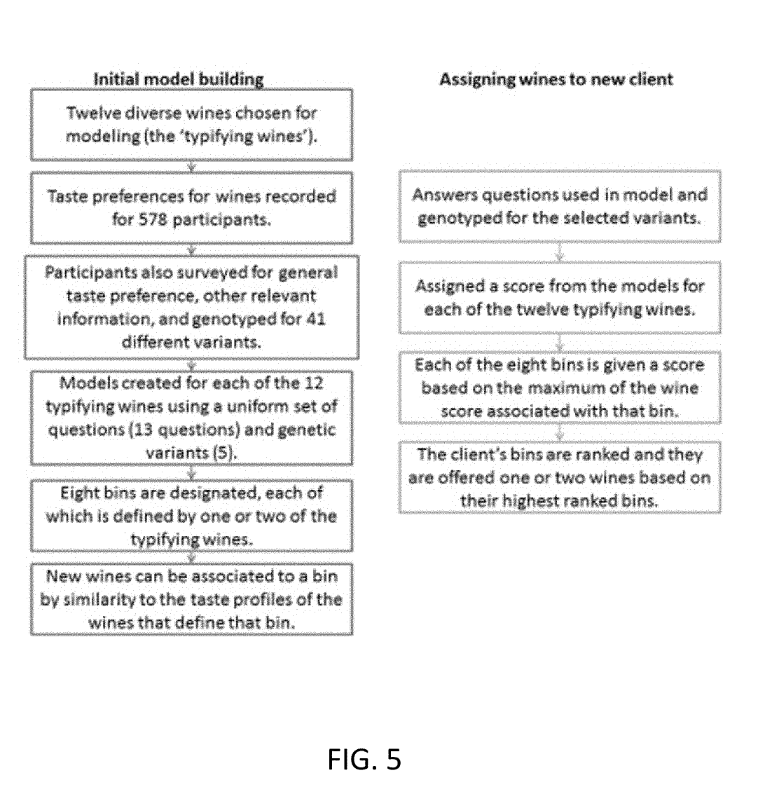

The basic process via which subjects are offered a wine is shown in FIG. 5. In short, using the models for twelve diverse wines established with the initial study population, subjects are given a score for the twelve wines used to describe a wide variant of wine preferences, and then given a score for a set of eight wine ‘bins’ that describe broad categories of wine preferences. The wine bin scores are ranked, and bins that are likely to be preferred will have higher scores and less preferred bins.

Wines were grouped according to eight bins defined by hierarchical clustering (Table 3), and a person's score for each bin was equal to that given by the model for the wine associated with that bin which had the highest score. For example, if an individual's score for Grenache was 1.2 and for the Zinfandel was 2.4, the individual's score for the “lighter chocolaty raspberry reds” bin, which those two wines are associated with, would be 2.4. Association of individual bin scores with actual averaged preference for the wines assigned to the bin was measured using ordered logistic regression (Table 4). All models were found to be highly significant.

Wines are assigned to the subject by ranking the bin scores, and offering the subject a wine from the highest ranked bin. If the other bins have negative rankings, no other wines are offered. If the other bins are positive, that is, the subject has a higher than average preference for wines of those bins, then another wine is offered. If the lowest ranked bin of the same wine color (i.e. red or white) as the highest ranked bin is greater than the highest ranked bin of the opposite color, then a wine of the second highest ranked bin is offered. Otherwise, a wine of the highest ranked bin of the opposite color is offered.

These results showed that methods and systems of the invention are useful for predicting wine preferences in a subject. These results further suggested that the methods and systems of the present invention are useful for creating a taste profile for a subject and providing a wine to a subject based on the subject's taste profile.

TABLE 3
Wine Models and Their Association with Bins.

| Bin | typifying wine |
|---|---|
| sweet whites | Radius Riesling 2013 |
| crisp and citrusy | Kim Crawford Sauv BI |
| whites | |
| crisp and floral | Cliff Lede Sauv BI |
| whites | |
| unoaked full- | Mer Soleil Chard |
| bodied whites | |
| oaked full-bodied | Sbragia Chard |
| whites | Rombauer Chard |
| light, cherry reds | Thumbprint Pinot |
| | Noir |
| | Siduri Pinot Noir |
| light chocolaty | Grenache |
| raspberry reds | Zinfandel |
| bold smoky | Syrah |
| blackberry reds | Cab Sauv |

TABLE 4
Testing of Predicted Bin Preferences.

| | effect | |
|---|---|---|
| | (95% CI) | P value |
| sweet whites | 0.52 | 1.55E−06 |
| | (0.31, 0.73) | |
| crisp and citrusy whites | 0.43 | 0.000321 |
| | (0.2, 0.66) | |
| crisp and floral whites | 0.61 | 4.42E−08 |
| | (0.39, 0.83) | |
| unoaked full-bodied whites | 0.58 | 0.00067 |
| | (0.25, 0.91) | |
| oaked full-bodied whites | 0.69 | 1.21E−06 |
| | (0.41, 0.97) | |
| light, cherry reds | 0.7 | 2.37E−11 |
| | (0.5, 0.91) | |
| light chocolaty raspberry | 0.58 | 6.99E−09 |
| reds | (0.39, 0.78) | |
| bold smoky blackberry reds | 0.61 | 2.96E−12 |
| | (0.44, 0.79) | |

### Example 2: Characterization and Classification of Wine Lists and Selection of Food and Wine Based on Genetic Profiles

Characterization and classification of wine lists and selection of food and wine based on genetic profiles is performed as follows. Adult participants are surveyed concerning their taste preferences, eating and drinking habits, and relevant demographic questions. Saliva samples are obtained from each participant and the samples are genotyped at a plurality of single nucleotide variants (SNPs) in a panel of SNPs identified as useful for identifying taste preferences. Variants are chosen by literature review to identify a variety of alleles with likely association with differences in the ability to perceive certain tastes.

Wines are analyzed using gas chromatography mass spectrometry (GC-MS). GC-MS data is used to catalog various wines and match wine to taste preferences. A computer implemented method described herein is used to recommend food and wine pairings to an individual based on the individual's taste preferences and wine catalog and wine characteristics.

These results show that the methods of the invention can be used to provide wine and food pairings to individuals based on their predicted taste and/or scent preferences. These results further show that the methods of the present invention may be used to provide gustative and olfactive products to consumers based on their predicted taste and/or scent preferences.

### Example 3: Genetic Profiling to Predict Individual Taste Preferences and Cocktail Selection Based on Genetic Profiles

Genetic profiling to predict individual cocktail taste preferences and selection of cocktails based on genetic profiles is performed as follows. Adult participants are surveyed concerning their taste preferences, eating and drinking habits, and relevant demographic questions. Saliva samples are obtained from each participant and the samples are genotyped at a plurality of single nucleotide variants (SNPs) in a panel of SNPs identified as useful for identifying taste preferences. A taste profile is generated for each participant based on the genotyping results. A computer implemented method described herein is used to recommend cocktails and cocktail recipes to an individual based on the individual's taste preferences and taste profile.

These results show that the methods of the invention can be used to provide cocktail recommendations to individuals based on their predicted taste and/or scent preferences. These results further show that the methods of the present invention may be used to provide gustative and olfactive products to consumers based on their predicted taste and/or scent preferences.

### Example 4: Genetic Profiling to Predict Tolerance for Bitter, Sour, and Spicy Tastes

Genetic profiling to predict tolerance for bitter, sour, and spicy tastes is performed as follows. Adult participants are surveyed concerning their taste preferences, eating and drinking habits, and relevant demographic questions. Saliva samples are obtained from each participant and the samples are genotyped at a plurality of single nucleotide variants (SNPs) in a panel of SNPs identified as useful for identifying taste preferences and tolerance for bitter, sour, and spicy tastes. A taste profile is generated for each participant based on the genotyping results. A computer implemented method described herein is used to recommend gustative products (e.g., hot sauces, beer, or coffee) to an individual based on the individual's taste preferences and taste genetic profile.

These results show that the methods of the invention can be used to provide gustative product recommendations to individuals based on their predicted taste preferences. These results further show that the methods of the present invention may be used to provide gustative and olfactive products to consumers based on their predicted taste and/or scent preferences.

### Example 5: Genetic Profiling to Predict Food Preferences for Babies

Genetic profiling to predict food preferences for babies is performed as follows. Saliva samples are obtained from each baby and the saliva samples are genotyped at a plurality of single nucleotide variants (SNPs) in a panel of SNPs identified as useful for identifying taste preferences, food sensitivities (e.g., gluten sensitive), and tolerance for foods (e.g., lactose intolerance). A taste profile is generated for each baby based on the genotyping results which predicts tastes that the baby is likely to enjoy. A computer implemented method described herein is used to recommend gustative products for the baby based on the baby's taste genetic profile.

These results show that the methods of the invention can be used to provide gustative product recommendations to babies based on their predicted taste preferences. These results further show that the methods of the present invention may be used to provide gustative and olfactive products to consumers based on their predicted taste and/or scent preferences.

### Example 6: Genetic Profiling to Predict Scent Preferences

Genetic profiling to predict individual scent preferences is performed as follows. A saliva sample is obtained from the individual and the saliva sample is genotyped at a plurality of single nucleotide variants (SNPs) in a panel of SNPs identified as useful for identifying scent preferences. A scent profile is generated for the individual based on the genotyping results which predicts scents that the individual is likely to enjoy or reject. A computer implemented method described herein is used to recommend olfactive products for the individual based on the individual's scent genetic profile.

These results show that the methods of the invention can be used to provide olfactive product recommendations to individuals based on their predicted scent preferences. These results further show that the methods of the present invention may be used to provide gustative and olfactive products to consumers based on their predicted taste and/or scent preferences.

### Example 7: Genetic Profiling to Predict Liquor and Other Beverage Taste Preferences

Genetic profiling to predict individual alcohol and other beverage taste preferences was performed as follows. Adult participants were surveyed concerning their taste preferences, eating and drinking habits, and relevant demographic questions. A total of 578 questions were asked. The surveys were conducted prior to and at nine different wine tasting events, with 541 people surveyed in total. SurveyMonkey.com was used to digitally collect survey responses from participants. Informed consent was obtained from all study participants.

Study participant were genotyped for the forty-one (41) single nucleotide variants (SNPs) listed in Table 1. Variants were chosen by literature review to identify a variety of alleles with likely association with differences in the ability to perceive certain tastes. Saliva was collected from survey participants using a collection kit (DNA Genotek, Ottawa, Canada) and genomic DNA isolated using the NA Genotek PrepIT*L2P saliva protocol as per manufacturers recommendation. Genotyping was performed by the HudsonAlpha Institute for Biotechnology (Huntsville, Ala.). Briefly, genomic DNA quantity was measured using Invitrogen's PicoGreen assay. Taqman custom and prepared SNP assays were obtained from Applied Biosystems/Thermo. Assays were provided as a probe/primer mix, lyophilized in a 384-well plate. Assays were plated by Thermo in duplicate, with four sets of assays loaded per plate. Genomic DNA samples were mixed with an equivalent amount of 2×master mix in a sterile reservoir, and the mixture was then aliquoted with a Biomek FxP robot to the 384-well plates. The final reaction volume of each well was 5 PCR reactions were run using manufacturer recommended conditions on an ABI thermal cycler (40 cycles). Completed reactions were analyzed using the Applied Biosystems QuantStudio 12K flex system.

Survey data was encoded numerically, with taste preferences encoded as −1 to +1, representing “dislike”, “no preference”, and “like”. Other questions were similarly encoded (Table 5). Genotypes were encoded as 0, 1, or 2 for the major homozygous, heterozygous, and minor homozygous alleles, respectively.

TABLE 5
Coding of survey questions for numerical analysis

| question | value | meaning |
|---|---|---|
| taste_pref.x | −1 | dislike |
| | 0 | neutral |
| | 1 | like |
| specific_tastes.x | 0 | disagree |
| | 1 | agree |
| savory_vs_sweet | −1 | sweet |
| | 0 | neutral |
| | 1 | savory |
| cheese_pref.x | 0 | don't prefer |
| | 1 | prefer |
| spices.x | 0 | don't prefer |
| | 1 | prefer |
| drink_alcohol.response | 0 | no |
| | 1 | yes |
| alcohol_pref.x | 0 | don't prefer |
| | 1 | prefer |
| varietal_pref.x | 0 | don't prefer |
| | 1 | prefer |
| light_vs_full.response | 0 | light |
| | 1 | full |
| sweet_vs_oak.response | 0 | sweet |
| | 1 | oak |
| smoke.response | 0 | never |
| | 1 | previously |
| | 2 | currently |
| age.response | 1 | under 21 |
| | 2 | 21-30 |
| | 3 | 31-40 |
| | 4 | 41-50 |
| | 5 | 51-60 |
| | 6 | 60 or older |
| | 7 | Decline to answer |
| gender.response | 1 | male |
| | 2 | female |
| SNP (rsnnnn) | 0 | homozygous major allele |
| | 1 | heterozygous |
| | 2 | homozygous minor allele |

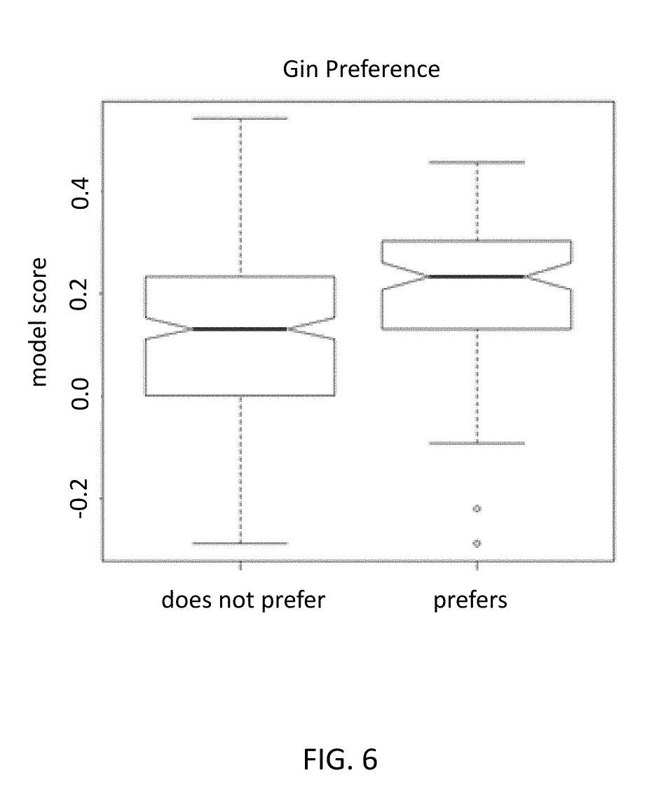

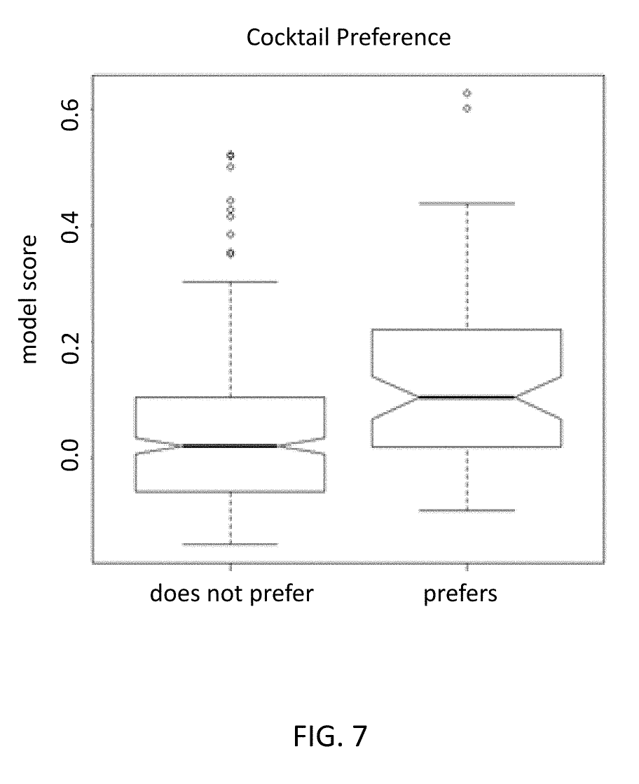

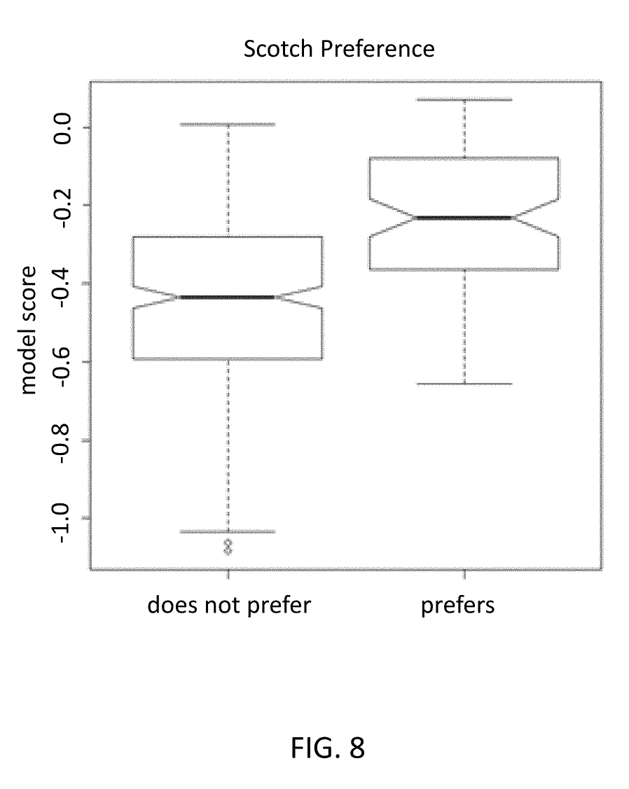

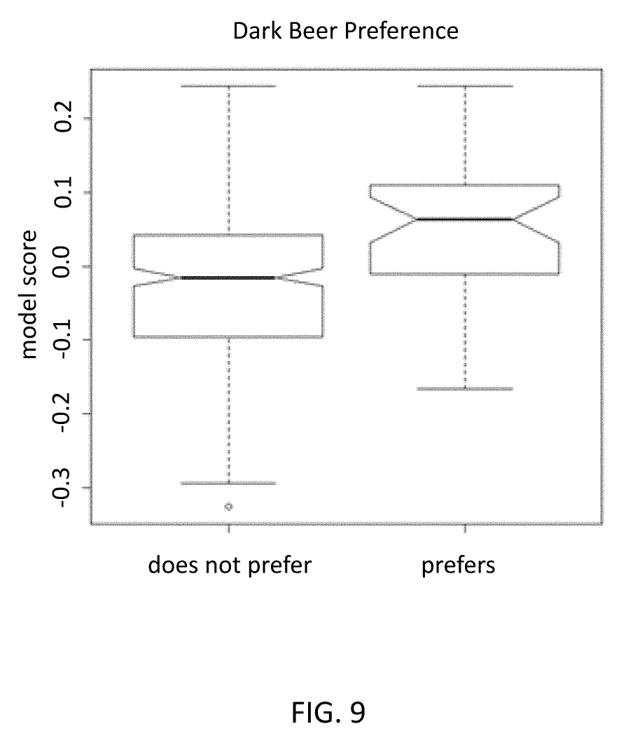

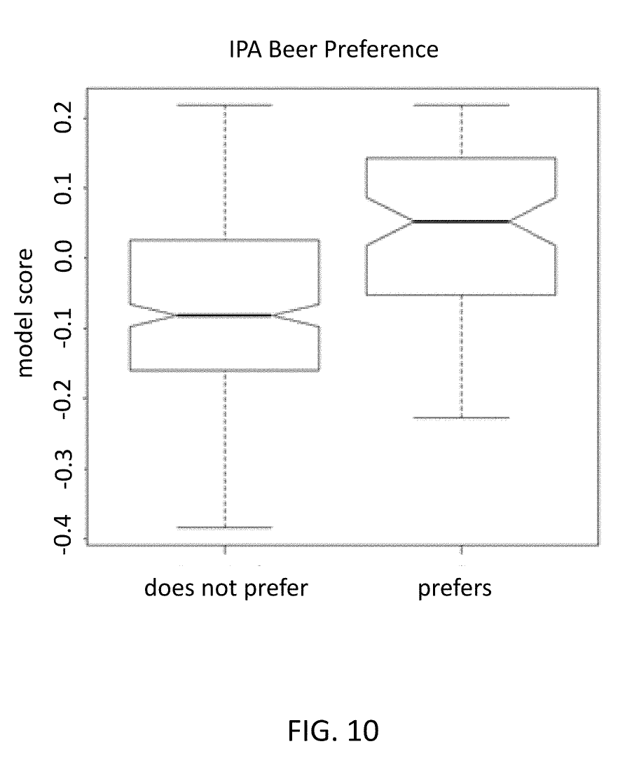

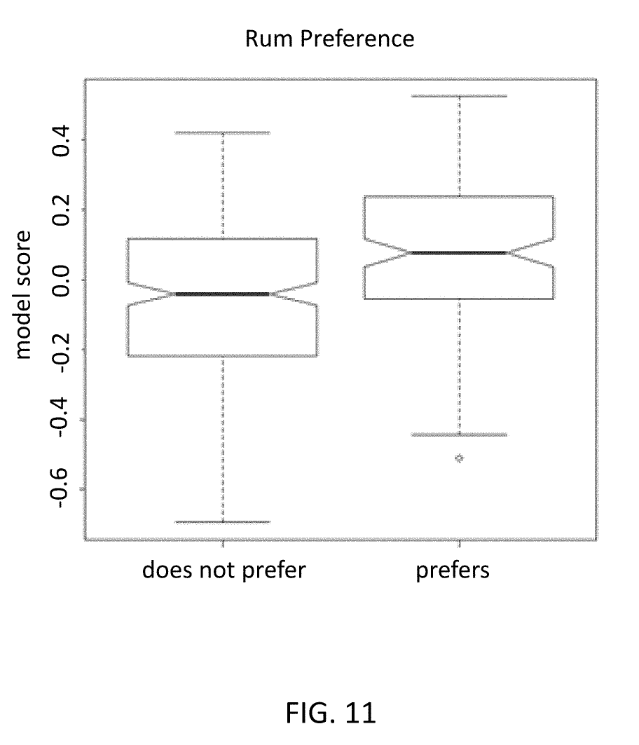

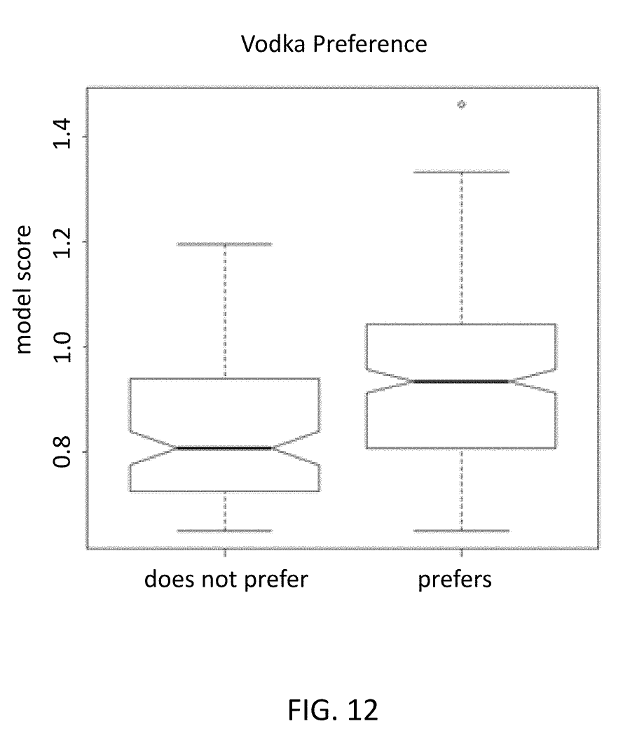

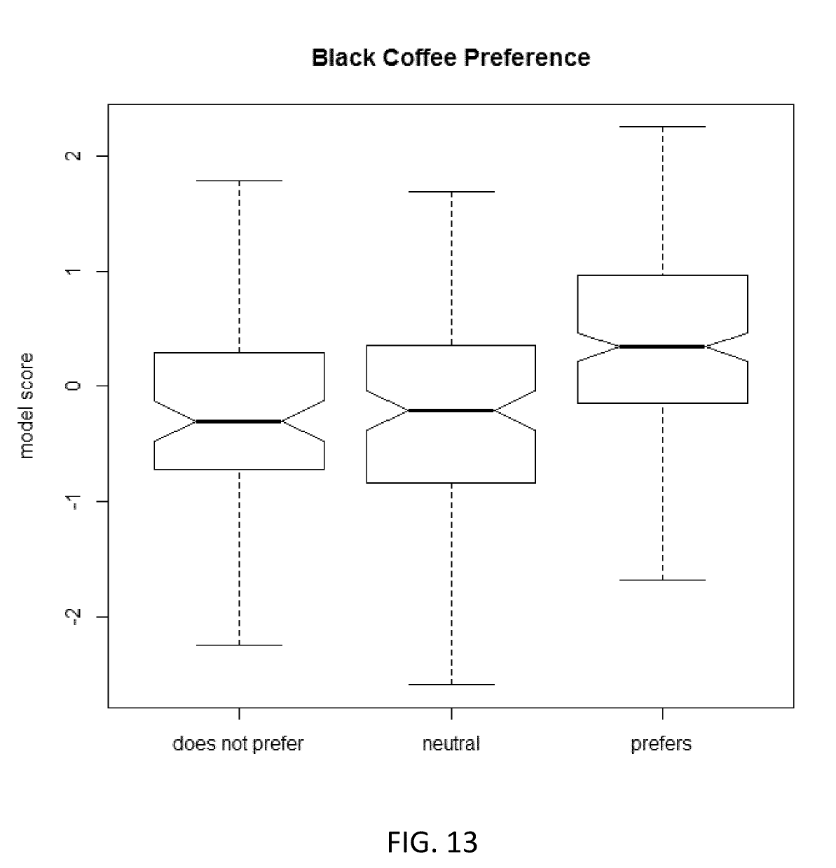

Variables for models predicting preference for the selected beverages were chosen from those survey responses and genetic variants having the greatest effect size, for which the 95% confidence intervals were both positive or both negative, and which showed a consistent relationship between the dependent variables. The effect size from the ordered logistic regression was used as the coefficient for the variables in the beverage preference models. Predicted preference for the beverage in question was modeled as β₁ρ₁+β₂ρ₂+ . . . where βₙ represents the model coefficient and ρₙ is the survey or genotype score. Model outcome was tested using a Student's T-test (Table 6). T-score calculations for the models are shown in Table 7. All models were found to be highly significant for alcohol and other beverage taste preferences. The distribution of scores are shown in FIGS. 6-13.

TABLE 6
Model coefficients

| | | effect | |
|---|---|---|---|
| predicted | predictor | size | p value |
| Liquor | spices.vanilla | −0.16 | 0.0000 |
| preference: | spices.garlic | 0.13 | 0.0017 |
| Gin | cheese_pref.american | −0.13 | 0.0469 |
| | cheese_pref.brie | 0.10 | 0.0065 |
| | rs2708377 | 0.09 | 0.0453 |
| | rs10772420 | 0.07 | 0.0238 |
| Alcohol | varietal_pref.sweet.sparkling | 0.28 | 0.0004 |
| preference: | cheese_pref.american | 0.14 | 0.0182 |
| Cocktails | spices.pepper | −0.09 | 0.0071 |
| | rs4920566 | 0.08 | 0.0022 |
| | spices.vanilla | 0.08 | 0.0220 |
| | tastes_pref.leather | −0.06 | 0.0455 |
| Liquor | gender.response | −0.22 | 0.0000 |
| preference: | varietal_pref.sweet.sparkling | −0.19 | 0.0259 |
| Scotch | spices.vanilla | −0.15 | 0.0001 |
| Whiskey | rs4684677 | −0.14 | 0.0169 |
| | cheese_pref.blue | 0.08 | 0.0367 |
| | rs4595035 | −0.07 | 0.0175 |
| | tastes_pref.flavored_coffee | −0.07 | 0.0049 |
| | tastes_pref.leather | 0.06 | 0.0419 |
| | tastes_pref.black.coffee | 0.08 | 0.0003 |
| Beer | tastes_pref.leather | 0.081591 | 0.000641 |
| preference: | liquor.pref.Scotch.whiskey | 0.07022 | 0.036493 |
| Dark | gender.response | −0.06709 | 0.016624 |
| | tastes_pref.black.coffee | 0.059984 | 0.000846 |
| | rs2277675 | −0.05019 | 0.027852 |
| | rs2274333 | 0.049523 | 0.031369 |
| Taste | varietal_pref.sweet.sparkling | −1.0026 | 0.004619 |
| Preference: | liquor.pref.Scotch.whiskey | 0.768205 | 0.000307 |
| Black Coffee | tastes_pref.leather | 0.604823 | 2.10E−05 |
| | tastes_pref.blackberries | 0.517397 | 0.000393 |
| | spices.vanilla | −0.43211 | 0.009995 |
| | cheese_pref.cheddar.sharp | 0.418017 | 0.014567 |
| | gender.response | −0.40595 | 0.015161 |
| | rs42451 | 0.304114 | 0.039064 |
| | rs838133 | −0.25992 | 0.048879 |
| Beer | specific_tastes.broccoli.bitter | −0.0962 | 0.0283 |
| preference: | spices.vanilla | −0.0850 | 0.0181 |
| IPA | tastes_pref.black.coffee | 0.0756 | 0.0010 |
| | tastes_pref.flavored_coffee | −0.0746 | 0.0011 |
| | tastes_pref.pungent.cheeses | 0.0674 | 0.0034 |
| | rs6591536 | −0.0604 | 0.0397 |
| Liquor | rs4684677 | 0.1835 | 0.0061 |
| preference: | spices.vanilla | 0.1611 | 0.0001 |
| Rum | rs28757581 | −0.1135 | 0.0217 |
| | rs9295791 | −0.1105 | 0.0463 |
| | drink_alcohol_freq.response | −0.1094 | 0.0002 |
| | tastes_pref.sweet_coffee | 0.1025 | 0.0001 |
| | tastes_pref.sweet.desserts | 0.0757 | 0.0152 |
| Liquor | drink_alcohol.response | 0.6955 | 0.0002 |
| preference: | gender.response | 0.1572 | 0.0001 |
| Vodka | rs9295791 | 0.1322 | 0.0150 |
| | light_vs_full.response | −0.1276 | 0.0027 |
| | varietal_pref.chardonnay | 0.1103 | 0.0171 |
| | tastes_pref.flavored_coffee | 0.0753 | 0.0041 |

TABLE 7
Model Testing

| model | t score | p value |
|---|---|---|
| Liquor preference: Gin | −5.5454 | 1.04E−07 |
| Alcohol preference: Cocktails | −4.5568 | 1.57E−05 |
| Liquor preference: Scotch Whiskey | −8.1178 | 1.06E−13 |
| Beer preference: Dark | −3.9258 | 0.00028 |
| Taste Preference: Black Coffee | −5.8537 | 3.00E−08 |
| Beer preference: IPA | −6.9347 | 1.47E−10 |
| Liquor preference: Rum | −6.1392 | 3.31E−09 |
| Liquor preference: Vodka | −6.5495 | 4.07E−10 |

These results showed that methods and systems of the present invention are useful for predicting alcohol, beer, and coffee preferences in a subject. These results further suggested that the methods and systems of the present invention are useful for creating a taste profile for a subject and providing liquor, beer, or coffee to a subject based on the subject's taste profile. These results further showed that the methods and systems of the present invention are useful for identifying taste and/or scent preferences for a subject. These results showed that the methods of the present invention are useful for selecting a gustative product for a subject based on the taste profile of the subject.

While the preferred embodiments of the invention have been illustrated and described, it will be appreciated that various changes can be made therein without departing from the spirit and scope of the invention.

What is claimed is:

1. A method comprising: a) obtaining a biological sample comprising nucleic acids from a subject; b) processing the sample to isolate or enrich the sample for the nucleic acids; and c) analyzing the genotype of the biological sample at a plurality of single nucleotide polymorphisms (SNPs) in a panel of SNPs identified as useful for identifying taste and/or scent preference.

2. The method of claim 1, further comprising obtaining taste and/or scent preference information from the subject.

3. The method of claim 1, further comprising calculating gustative and/or olfactive product bin scores based on the genotype of the sample.

4. The method of claim 1, wherein the plurality of SNPs is selected from the group consisting of rs838133, rs1421085, rs3930459, rs2274333, rs111615792, rs72921001, rs7792845, rs10772420, rs846672, rs9295791, rs6591536, rs1761667, rs10772397, rs586965, rs5020278, rs11988795, rs4595035, rs12226920, rs1524600, rs61729907, rs1548803, rs4684677, rs42451, rs35680, rs34911341, rs696217, rs26802, rs2708377, rs28757581, rs35744813, rs4920566, rs3935570, rs2277675, rs71443637, rs9276975, rs34160967, rs307377, rs713598, rs1726866, rs10246939, and rs846664.

5. The method of claim 1, wherein the plurality of SNPs comprises at least 5, 10, 15, 20, 25, 30, 35 or 41 SNPs selected from Table 1.

6. The method of claim 1, further comprising determining the subject's taste and/or scent preference scores for one or more food or beverage product.

7. A method for predicting taste and/or scent preferences of a subject, the method comprising: a) providing a biological sample comprising DNA from the subject; b) genotyping a plurality of SNPs in the sample, wherein the plurality of SNPs comprises one or more SNPs identified as useful for identifying taste and/or scent preference; c) obtaining taste and/or scent preference information from the subject; and d) determining the subject's taste and/or scent preference scores for one or more food or beverage product, thereby predicting taste and/or scent preferences for the subject for a gustative or olfactive product.

8. The method of claim 7, further comprising calculating gustative and/or olfactive product bin scores based on the genotyping from step (b), the information obtained about the subject from step (c), and the taste and/or scent preference scores for the one or more gustative or olfactive products from step (d).

9. The method of claim 8, wherein the gustative or olfactive product bins comprise one or more gustative or olfactive product bins.

10. The method of claim 8, the gustative or olfactive bin scores are used to predict that the subject will prefer gustative or olfactive products from a gustative or olfactive product bin having a higher gustative or olfactive product bin score than a gustative or olfactive product bin having a lower gustative or olfactive product bin score.

11. The method of claim 8, further comprising creating and storing a user taste and/or scent profile comprising information about the subject's gustative and olfactive product preferences based on the subject's gustative and/or olfactive product bin scores.

12. The method of claim 11, further comprising providing the subject with at least one sample of a gustative or olfactive product from the gustative or olfactive bin having the highest gustative or olfactive product bin score.

13. The method of claim 7, wherein the food or beverage product is selected from the group consisting wine, liquor, beer, and coffee.

14. The method of claim 13, wherein the liquor is selected from the group consisting of gin, scotch whiskey, rum, and vodka.

15. A method for providing a gustative and/or olfactive product to a subject, the method comprising the following steps: (a) providing a biological sample comprising DNA from the subject; (b) genotyping the sample to determine which alleles are present at a set of single nucleotide polymorphisms identified as useful for identifying taste and/or scent preference; (c) obtaining information about the subject; (d) determining the subject's taste and/or scent preference scores for one or more gustative and/or olfactive products; and (e) calculating gustative and/or olfactive product bin scores for one or more gustative and/or olfactive product bins based on the genotyping from step (b), the information obtained about the subject from step (c), and the taste and/or scent preference scores for one or more gustative and/or olfactive products from step (d); and (f) providing the subject with at least one gustative and/or olfactive product from the gustative and/or olfactive product bin that has the subject's highest gustative and/or olfactive product bin score.

16. The method of claim 15, further comprising creating and storing a user taste and/or scent profile comprising information about the subject's gustative and/or olfactive product preferences based on the subject's gustative and/or olfactive product bin scores.

17. The method of claim 16, further comprising providing the subject with at least one sample of gustative and/or olfactive product from the gustative and/or olfactive product bin having the highest gustative and/or olfactive product bin score.

18. The method of claim 15, wherein the food or beverage product is selected from the group consisting of wine, liquor, beer, and coffee.

19. The method of claim 18, wherein the liquor is selected from the group consisting of gin, scotch whiskey, rum, and vodka.
